(19) United States
(12) Patent Application Publication
(10) Pub. No.: US 2009/0305233 A1
(43) Pub. Date: Dec. 10, 2009

(54) Methods and Reagents for Polynucleotide Assembly

(75) Inventors: Alexandre Yurievich Borovkov, Tempe, AZ (US); Kathryn Frances Sykes, Tempe, AZ (US); Andrey Vasilievich Loskkutov, Higley, AZ (US)

Correspondence Address: Baker Donelson Bearman, Caldwell & Berkowitz, PC, 555 Eleventh Street, NW, Sixth Floor, Washington, DC, 20004, US

(73) Assignee: Arizona Board of Regents, a body corporate of the State of Arizona

(21) Appl. No.: 11/849,608

(22) Filed: Sep. 4, 2007

Related U.S. Application Data
(60) Provisional application No. 60/958,305, filed on Jul. 3, 2007.

(51) Int. Cl. C12Q 1/68; C12P 19/34; C40B 40/06; C07H 21/00

(52) U.S. Cl. 435/6; 435/91.2; 506/16; 536/25.3

(57) ABSTRACT

The present invention provides methods, compositions, and kits for polynucleotide assembly.

### CROSS REFERENCE

This application claims priority to U.S. Provisional Patent Application Ser. No. 60/958,305 filed Jul. 3, 2007, incorporated by reference herein in its entirety.

### STATEMENT OF GOVERNMENT INTEREST

This invention was made with government support under grant nos. 1 PO1 AI056295-01AI and U54AI057156 awarded by the National Institute of Health. The government has certain rights in the invention.

### BACKGROUND OF THE INVENTION

There is a growing need for synthetic gene assembling, but none of the currently available protocols [1-22] is suitable for robust and reliable high throughput applications. Most are based on the PCR-based (oligo)nucleotides assembling approach developed more then ten years ago, and rely heavily on the quality of the oligonucleotides used in the assembly process, leading to very high error rates (85-95% or more), and thus requiring extensive analysis of assembled genes to identify those assembled accurately.

### SUMMARY OF THE INVENTION

The present invention provides methods, compositions, and kits for polynucleotide assembly. In a first aspect the present invention provides methods for polynucleotide assembly comprising:
(a) incubating, under hybridizing conditions, an oligonucleotide pool comprising a plurality of assembly oligonucleotides having mutually reverse complementary overlap segments, wherein the incubating results in assembly of a plurality of fully or partially double stranded polynucleotide assembly blocks, wherein the incubation is performed under conditions favoring hybridization of overlap segments that are exactly reverse complementary one to the other over hybridization of overlap segments with oligonucleotides whose sequences are not exactly reverse complementary thereto;
(b) amplifying the polynucleotide assembly blocks; and
(c) amplifying a polynucleotide of interest from one or more polynucleotide assembly blocks, wherein adjacent polynucleotide assembly blocks are joined at regions of mutual overlap as necessary to produce the polynucleotide of interest.

In a second aspect, the present invention provides a polynucleotide assembly block amplification pool, comprising a plurality of polynucleotide assembly blocks which each comprise adaptor sequences permitting general or selective amplification of polynucleotide assembly blocks from the pools.

In a third aspect, the present invention provides a polynucleotide assembly block stock pool, comprising a plurality of polynucleotide assembly blocks having regions of common sequence, thus permitting selective amplification of a polynucleotide of interest.

In a fourth aspect, the present invention provides an oligonucleotide pool comprising a plurality of overlapping assembly oligonucleotides, wherein the overlap segments of the plurality of assembly oligonucleotides have lengths and compositions fostering preferential hybridization of overlap segments that are exactly reverse complementary one to the other over hybridization of overlap segments with oligonucleotides whose sequences are not exactly reverse complementary thereto.

In a fifth aspect, the present invention provides kits, comprising one or more of the compositions of the invention.

In a sixth aspect, the present invention provides methods and machine readable storage media for oligonucleotide design, and described in detail below.

### BRIEF DESCRIPTION OF THE FIGURES

FIG. 1 is an illustrative example of the assembly of assembly oligonucleotides into polynucleotide assembly blocks and polynucleotide assembly blocks into a target polynucleotide.

FIG. 2(a-c) is an illustrative example of the assembly of assembly oligonucleotides. (a) If the assembly oligonucleotides are fully overlapping, the resulting hybridized complex spans the entire sequence of both strands. (b) If the assembly oligonucleotides are partially overlapping, single-stranded gaps may exist in the resulting complex. (c) One or more assembly oligonucleotides may also contain additional sequences overlapping or not the adjacent assembly oligonucleotide of the same sense strand. (I think it is more important to emphasize that extra sequences don't have to be complimentary to the target. They can be common between oligos and used as hybridization anchors during purification, as amplification prime sites, or for some other manipulations with oligos prior to their assembling. Simple redundancy of the sequence is also acceptable, but I personally don't see why someone will do it.).

FIG. 3 is an illustrative example of the temperature effect on duplex formation between perfectly matching and minimally mismatching oligonucleotides.

FIG. 4(a-g) is an exemplary schematic illustration of LCR assembly resulting in the hybridization and ligation of a plurality of overlapping assembly oligonucleotides into intermediate fragments and intermediate fragments into complete polynucleotide assembly blocks. (a) The assembly oligonucleotides corresponding to a polynucleotide assembly block are initially present in the assembly oligonucleotide pool and the temperature is raised to ensure that all duplexes are melted. (b) In the first cycle of annealing complementary regions of the assembly oligonucleotides associated into duplexes and abutting ends are ligated ((c) circles). In the next cycle of melting the duplexes dissociate (d) and on reannealing duplexes again form (e) and are ligated ((e), circles). In another cycle of melting (f) and annealing and ligating (g) the complete polynucleotide assembly block is formed.

FIG. 5(a-f) is an example (together with FIG. 6) of assembling polynucleotide assembly blocks using spPCR. The assembly oligonucleotides corresponding to a polynucleotide assembly block are initially present in the assembly oligonucleotide pool and the temperature is raised to ensure that all duplexes are melted (a). In the first cycle of annealing complementary regions of the assembly oligonucleotides associated into partially overlapping (b) and extended into fully double stranded duplexes (c). Duplexes are melted (d), and then new duplexes are allowed to form and again extended (e). An additional cycle of melting is carried out (f); the remaining steps are illustrated in FIG. 6.

FIG. 6 (a-c) completes the example shown in FIG. 5. After carrying out the additional cycle of melting in FIG. 5(f), annealing and extending (a) results in yet larger intermediate fragments. The duplexes are again melted (b) and single stranded fragments annealed and the 3′ overlaps are extended, producing a complete polynucleotide assembly block (c).

FIG. 7 illustrates schematically the composition of the polynucleotide assembly blocks during their assembling and amplification (a) and after removing of the adapter regions (b).

FIG. 8 (a-f) illustrates schematically an embodiment of a method for joining and amplifying a target polynucleotide SEQ from two adjacent polynucleotide assembly blocks. (a) Two polynucleotide assembly blocks comprise block overlap regions BOR of identical sequence. (b-c) Upon melting and annealing of the sequence specific primers 706, 707, the polymerase extends from the primers, resulting in overlapping single strands of opposite sense, spanning the sequence SEQ of the target polynucleotide. (d) Upon melting, followed by annealing of the overlapping strands, the polymerase extends from the overlap using the opposite sense strands as templates, resulting in a double-stranded polynucleotide having the desired sequence (e), which can then be PCR amplified (f) using the sequence specific primers 706, 707.

FIG. 9 illustrates one example for removal of adaptors during block joining and amplification of a target polynucleotide, by using a thermostable polymerase having 3′ to 5′ exonuclease activity for overlap PCR joining of adjacent polynucleotide assembly blocks.

FIG. 10 (a-h) illustrates an example for removal of adaptor sequences during overlap joining and amplification by using a thermostable polymerase having 5′ to 3′ exonuclease activity. (a) Upon hybridization of the block overlap 186 of the antisense strand of the first polynucleotide assembly block 180 with the block overlap 187 of the sense strand of the second polynucleotide assembly block 183, single stranded 5′ terminal sequences complementary to the adaptor sequences will be exposed when present. (b-c) During extension of primers 192 and 194 the overlapping region will be displaced by 5′>3′ activity of the polymerase. In the process the regions 181 and 184 will be removed completely and regions 186, 187 partially, until destabilization of the hybrid. The resulting synthesized strands 196, 197 (d-e) retain some region of overlap which allows them anneal to each other (f). The chains are extended from the overlaps using the opposite sense strand as templates and the overlap as primers, producing the full length polynucleotide spanning the entire sequence between the forward and reverse primers (g-h).

FIG. 11 (a-g) shows results obtained in testing the influence of pool complexity on specificity of polynucleotide assembly, as described in Example 1.

FIG. 12 (a-b) provides data demonstrating that assembly oligonucleotides can be successfully assembled into polynucleotide assembly blocks even when the assembly oligonucleotides to be assembled are present at concentrations well below those currently practiced in the art. (a) Data from fluorescent readout of qPCR reactions described in Example 2, where X-axis is cycle number, and Y-axis is relative fluorescence units of the PCR generated product. (b) Photograph of stained agarose gel after electrophoresis to visualize the results.

FIG. 13 (a-c) show data obtained from experiments using an assembly oligonucleotide pool comprising assembly oligonucleotides for the assembly of 20 distinct polynucleotide assembly blocks, using three different methods. (a) One step PCR method. (b) The two-step PCR method. (c) Two step LCR-PCR method.

FIG. 14 shows data from experiments demonstrating efficiency of ligation and feasibility of the LCR preassembly step for assembly of polynucleotide assembly blocks each from eight assembly oligonucleotides by LCR using decreasing concentrations of assembly oligonucleotide ranging from 4.25 uM down to 50 pM.

FIG. 15 is a diagram of polynucleotide block assembly from eight internal assembly oligonucleotides spanning the gene specific region (GSR in FIG. 8) plus two terminal assembly oligonucleotides, as detailed in example 5. Each terminal assembly oligonucleotide carried a sequence (UPR) complimentary to the primer for block co-amplification. Two adaptor sequence designs, one for block A and one for block B, were used to avoid unintended amplification of the short regions between adaptors of adjacent blocks, corresponding to the block overlapping region (BOR).

FIG. 16 (a) shows data on the efficiency of amplification of polynucleotide block assembled from variable amounts of polynucleotide assembling oligonucleotides (detailed in Example 5) as monitored by qPCR.

FIG. 16 (b) shows data on the efficiency of polynucleotide block overlap with and without removal of the adapter sequences (detailed in Examples 8, 9) as monitored by qPCR.

FIG. 17 shows data on efficiency of assembly of three different target polynucleotide sequences from 2 to 6 polynucleotide assembly blocks using nominal block sizes of 250 and 350 base pairs assembled from 8 and 10 assembly oligonucleotides respectively.

FIG. 18 diagrams one exemplary embodiment of a technique for designing sequences of assembly oligonucleotides for use in assembly of polynucleotide assembly blocks from which desired target polynucleotide sequences can be obtained.

FIG. 19 diagrams one exemplary embodiment of a technique for improving the difference between the lowest melting temperature for any window and the highest single-mismatch annealing temperature for any window.

FIG. 20 diagrams one exemplary embodiment of a technique for demarcating a recoded composite sequence into a series of contiguous overlap segments that number one plus an integer multiple of eight.

FIG. 21 illustrates schematically an exemplary demarcation of a recoded combined sequence into polynucleotide assembly blocks, assembly oligonucleotides, and overlap segments.

### DETAILED DESCRIPTION OF THE INVENTION

All publications, patents and patent applications cited herein are hereby expressly incorporated by reference for all purposes.

Within this application, unless otherwise stated, the techniques utilized may be found in any of several well-known references such as: Molecular Cloning: A Laboratory Manual (Sambrook, et al., 1989, Cold Spring Harbor Laboratory Press) and PCR Protocols: A Guide to Methods and Applications (Innis, et al. 1990. Academic Press, San Diego, Calif.).

### DEFINITIONS

As used herein, the singular forms “a”, “an” and “the” include plural referents unless the context clearly dictates otherwise. For example, reference to a “nucleic acid” means one or more nucleic acids.

As used herein, “Polynucleotide” refers to any fully or partially double stranded nucleic acid amenable to the assembly methods of the invention. A single “polynucleotide” can comprise one or multiple different double stranded nucleic acids of interest (ie: genes, cDNAs, non-coding nucleic acids, etc.).

As used herein, “Target sequence” means the sequence of a polynucleotide desired to be synthesized and capable of being synthesized using any one or more of the methods, compositions, and kits disclosed herein. “Recoded target sequence” refers to the sequence actually to be synthesized after recoding, if any, has been accomplished. “Unrecoded target sequence” refers to a sequence desired to be recoded and synthesized and capable of being recoded and synthesized using any one or more of the methods, compositions, and kits disclosed herein, as such sequence is originally specified prior to recoding. A target sequence can be a recoded or unrecoded target sequence or both, as deemed appropriate for a given use. As used herein with respect to a target sequence, the “sense” strand refers to the nucleic acid strand having the same sequence as the target sequence, regardless of whether or not the sequence encodes a protein or is otherwise expressed, and the “antisense” strand refers to the strand reverse complementary to the sense strand. “Target polynucleotide” means a polynucleotide desired to be synthesized and capable of being synthesized using any one or more of the methods, composition, and kits disclosed herein.

As used herein, “recode” means and includes any and/or all of the methods disclosed herein whose purpose or effect is to make one or more substitutions in a polynucleotide sequence of a codon type for another codon type, for the purpose of melting temperature adjustment, avoidance of undesired codon types, elimination of restriction sites, avoidance of undesired secondary structure, and/or any other purpose to which the disclosures hereof are addressed.

An “assembly oligonucleotide” is one of the plurality of overlapping single stranded oligonucleotides that are used to construct polynucleotide assembly blocks. The single stranded assembly oligonucleotides for a given double stranded polynucleotide assembly block include oligonucleotides for both strands, referred to herein as “sense” and “antisense” strands, solely to note their presence on opposite strands of the resulting polynucleotide assembly block, and not to imply that a coding sequence is required. Assembly oligonucleotides can be of any length sufficient to accommodate the overlap segments; preferably, each assembly oligonucleotide is at least about 15, 20, 25, 30, 35 or 40 nucleotides in length. The assembly oligonucleotides incorporated into a polynucleotide assembly block, and/or the assembly oligonucleotides in an assembly oligonucleotide pool, may be of identical sizes or may be of a distribution of sizes. Any number of assembly oligonucleotides can be assembled to form a polynucleotide assembly block, subject only to the limitations arising from the ever-increasing ability of available enzymes (ie: polymerases, ligases, etc.) to assemble larger numbers of assembly oligonucleotides to form the intermediate fragments and polynucleotide assembly blocks. In various embodiments, there may be at least about 4-20 or more oligonucleotides per polynucleotide assembly block. Assembly oligonucleotides can comprise or consist of any composition capable of being assembled, ligated, and amplified using the methods, compositions, and kits disclosed herein, including without limitation DNA, RNA, peptide nucleic acids (“PNA”), 2′-5′ DNA (a synthetic material with a shortened backbone that has a base-spacing that matches the A conformation of DNA, and will not normally hybridize with DNA in the B form, but will hybridize readily with RNA) and locked nucleic acids (“LNA”), nucleic acid-like structures, as well as combinations thereof and analogues thereof. Assembly oligonucleotides may comprise nucleic acid analogues, including, but not limited to aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. Assembly oligonucleotides may also comprise nucleic acid backbone analogues including, but not limited to, phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs), methylphosphonate linkages or alternating methylphosphonate and phosphodiester linkages (Strauss-Soukup (1997) Biochemistry 36:8692-8698), and benzylphosphonate linkages, as discussed in U.S. Pat. No. 6,664,057; see also Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). Assembly oligonucleotides may also comprise analogous forms of ribose or deoxyribose as are well known in the art, including but not limited to 2′ substituted sugars such as 2′-O-methyl-, 2′-fluoro- or 2′-azido-ribose, carbocyclic sugar analogs, α.-anomeric sugars, epimeric sugars such as arabinose, xyloses or lyxoses, pyranose sugars, sedoheptuloses, acyclic analogs and abasic nucleoside analogs such as methyl riboside. The oligonucleotides may also contain TNA (threose nucleic acid; also referred to as alpha-threofuranosyl oligonucleotides) (See, for example, Schong et al., Science 2000 Nov. 17, 290 (5495):1347-1351.) In some embodiments, assembly oligonucleotides are phosphorylated at their 5′ termini, to facilitate ligation chain reaction (“LCR”) assembly techniques.

As used herein, the “melting temperature” of a double-stranded nucleic acid means the temperature at which, in a solution in which said nucleic acid is present under the conditions of interest and at a concentration of interest, 50% of the molecules of said nucleic acid remain in double-stranded duplexes and 50% have denatured into single strands. As used herein, the “melting temperature” of a single-stranded nucleic acid means the melting temperature of a double-stranded nucleic acid formed by hybridizing the single-stranded nucleic acid to its exact reverse complement. As used herein, the “melting temperature” of a sequence means the melting temperature of a nucleic acid whose sequence is said sequence.

As used herein, the “single-mismatch annealing temperature” of a sequence means, under the conditions of interest in a solution comprising molecules of a first single-stranded nucleic acid having said sequence, and further comprising molecules of a second single-stranded nucleic acid, present in equimolar concentration to said first single-stranded nucleic acid, wherein the sequence of the second single-stranded nucleic acid is selected from the set of all single-stranded nucleic acids that can be specified by making any single nucleotide substitution in the exact reverse complement of the sequence of the first single-stranded nucleic acid, the temperature at which 50% of the molecules of the first single-stranded nucleic acid and the second single-stranded nucleic acid have formed duplexes, and the second single-stranded nucleic acid is the single-stranded nucleic acid belonging to said set whose selection results in the highest said temperature.

An “overlap segment” is a region of a first assembly oligonucleotide that is reverse complementary to an overlap segment of a second assembly oligonucleotide, where the first and second assembly oligonucleotides both correspond to the same polynucleotide assembly block. In some embodiments an overlap segment of an assembly oligonucleotide includes a terminus of the assembly oligonucleotide. In some embodiments an assembly oligonucleotide may have a non-overlapping terminal region interposed between an overlap segment and the adjacent terminus of the assembly oligonucleotide. In some embodiments where an assembly oligonucleotide comprises two overlap segments, the two overlap segments are contiguous one with the other; in other embodiments where an assembly oligonucleotide comprises two overlap segments, there is a region interposed between the two overlap segments. In some embodiments where an assembly oligonucleotide comprises two overlap segments, the two overlap segments are contiguous one with the other and together make up the entire sequence of an assembly oligonucleotide (such an assembly oligonucleotide being referred to herein as a “fully overlapping assembly oligonucleotide”). Overlap segments may be of any length operable for hybridization in accordance with the methods, compositions, and kits disclosed herein, and in various embodiments may be at least about 10, 12, 14, 16, 18, 20, 22, 24, or more nucleotides in length. In various preferred embodiments, overlap segments are sequence adjusted for preferential hybridization of their exact reverse complements.

A “terminal assembly oligonucleotide” is an assembly oligonucleotide that comprises a terminus or terminal overhang of the polynucleotide assembly block to which it corresponds. A terminal assembly oligonucleotide may comprise an adaptor sequence which, in the assembled polynucleotide assembly block prior to amplification, may comprise a single-stranded overhang.

An “internal assembly oligonucleotide” is an assembly oligonucleotide that does not comprise a terminus or terminal overhang of a polynucleotide assembly block. An internal assembly oligonucleotide comprises two overlap segments, one of which is reverse complementary to an overlap segment of a first other assembly oligonucleotide, and the other of which is reverse complementary to an overlap segment of a second other assembly oligonucleotide, wherein the first and second other assembly oligonucleotides each correspond to the strand of opposite sense to the strand to which the internal assembly oligonucleotide corresponds.

An “adaptor sequence” is a sequence that is wholly or partially reverse complementary to a primer for amplification of a polynucleotide assembly block. In some embodiments an adaptor sequence may comprise a restriction enzyme recognition sequence to facilitate removal of the adaptor sequence. In various exemplary embodiments, an adaptor sequence is not a part of a target sequence, and all or a plurality of polynucleotide assembly blocks comprise adaptors that are wholly or partially reverse complementary to the same primer sequence, facilitating simultaneous amplification of multiple polynucleotide assembly blocks. Primers for polynucleotide assembly block amplification and to which an adaptor sequence is wholly or partially reverse complementary are referred to herein as “block amplification primers”. It is also possible for one or both termini of a polynucleotide assembly block not to comprise an adaptor, in which case all or part of the polynucleotide assembly block may be amplified using primers corresponding to the target sequence or other sequence present at the termini of the polynucleotide assembly block or part thereof to be amplified. Adaptor sequences and/or the primer sequences to which they are wholly or partially reverse complementary may be unique to one polynucleotide assembly block, universal to all polynucleotide assembly blocks present in a pool, or common to a plurality of polynucleotide assembly blocks.

An “assembly oligonucleotide pool” is a mixture comprising four or more single stranded assembly oligonucleotide species (4, 5, 6, 7, 8, 9, 10, 1, 12, 13, 14, 15, 16, 17, 18, 19, 20, or more) that is sufficient to provide all assembly oligonucleotides for assembly of at least one polynucleotide assembly block. An assembly oligonucleotide pool may comprise sufficient assembly oligonucleotides for a single, or a plurality (2, 3, 4, 5, or more) of polynucleotide assembly blocks and/or one or more polynucleotides of interest (e.g., 2, 3, 5, 10, 50, 100, 500, 1000, etc.). Assembly oligonucleotide pools can be provided in any form, including without limitation frozen, in solution, or bound to a substrate such as, by way of non-limiting example only, a microarray, microfluidics array, or solid phase material of a column. The oligonucleotides in the assembly oligonucleotide pool may be produced or synthesized via any suitable technique known to those of skill in the art, including but not limited to array-based synthesis, chemical synthesis, and automated synthesis. An assembly oligonucleotide pool may comprise assembly oligonucleotides that are synthesized individually or in multiplex fashion or both. Assembly oligonucleotide pools may, in addition to assembly oligonucleotides, comprise other oligonucleotides, including without limitation oligonucleotides that result from errors in the synthesis of the assembly oligonucleotides present in the assembly oligonucleotide pool.

As used herein, “expression environment” means any expression system, organism, or other environment in which an expression product is produced with respect to a polynucleotide sequence, including without limitation and by way of example only, bacterial expression systems, yeast expression systems, in vitro translation systems, and the transcription and/or translation system present in any organism.

As used herein, a “mismatch-exclusion temperature characteristic” means and includes any one or more characteristics of a polynucleotide or polynucleotide sequence or part thereof (including without limitation an overlap segment) where said one or more characteristics relates to, provides a reasonable estimated measure of, or is reasonably predictive of the propensity of the polynucleotide sequence or part thereof to hybridize with sequences other than its exact reverse complement under the hybridization conditions, including without limitation annealing temperature, used for the assembly said sequence or part thereof from assembly oligonucleotides. Without limiting the generality of the foregoing, and by way of example only, uniformity of melting temperature over the length of a sequence is a mismatch-exclusion temperature characteristic; uniformity of melting temperatures of the overlap segments of assembly oligonucleotides with respect to their exact reverse complements is a mismatch-exclusion temperature characteristic; and any measure of the separation between the melting temperature of a sequence with respect to its exact reverse complement or the distribution of melting temperatures of a set of sequences each with respect to its reverse complement, and the single-mismatch annealing temperature of said sequence or the distribution of single mismatch annealing temperatures of said set of sequences, is a mismatch-exclusion temperature characteristic.

As used herein, “polynucleotide assembly block amplification pool” means a mixture or solution comprising a plurality of polynucleotide assembly blocks and/or polynucleotide contigs each comprising adaptors for amplifying said polynucleotide assembly blocks with primers. As such, it is a subset of a polynucleotide assembly block stock pool (see below). The adaptors may be universal adaptors for amplifying all of said plurality of polynucleotide assembly blocks in parallel, and/or there may be one or more adaptor pairs each common to one or more subsets of said plurality of polynucleotide assembly blocks, for selectively amplifying in parallel fashion any of said one or more subsets or any combination thereof.

As used herein, “polynucleotide assembly block stock pool” means a mixture or solution comprising a plurality of polynucleotide assembly blocks and/or polynucleotide contigs, where said polynucleotide assembly blocks have been amplified and rendered suitable for storage, aliquotting, assembly, and/or amplification of selected target polynucleotide sequences.

As used herein, “spPCR” or “self-priming PCR” means any reaction in which at least two nucleic acid strands of opposite sense hybridize at a mutually reverse complementary region and at least one strand is extended by a polymerase using the other strand as a template.

As used herein, “overlap PCR” means any reaction in which at least two nucleic acid strands of opposite sense hybridize at a mutually reverse complementary region and at least one strand is extended by a polymerase using the other strand as a template, wherein primers are provided complementary to each strand and bounding a region that includes the region of mutual reverse complementarity, and the sequence bounded by the primers is amplified.

### Methods, Compositions and Kits for Polynucleotide Assembly

The methods, compositions, and kits of the invention can be used for robust and accurate parallel polynucleotide assembly from a single oligonucleotide pool. In various aspects, the methods, compositions, and kits disclosed herein utilize a unique building block approach which enables assembly of error-free polynucleotides from shorter oligonucleotides, even when these are present in as low as femtomolar or lower concentrations, and even using an oligonucleotide pool having high error content such as is commonly generated by array-based oligo syntheses. The need for purifying the oligonucleotide pool to remove incorrectly synthesized sequences prior to assembly is obviated by various of the methods, compositions, and kits of the invention, individually and in combination, including without limitation the use of a novel sequence optimization method; the use of an assembly protocol whereby the assembly oligonucleotides are joined under conditions that strongly disfavor hybridization of overlap segments that are not exactly reverse complementary thus providing selective pressure to limit incorporation into the polynucleotide assembly blocks of incorrectly synthesized assembly oligonucleotides. As a result, amplification and/or purification of oligonucleotides prior to use is unnecessary, making possible the direct use of inexpensive, commercially available oligo pools produced by array-based synthesis, despite the very small quantities and high error content of the oligos comprising such oligo pools. In another aspect, the invention provides a practicable approach for obtaining one or more of a plurality of desired polynucleotides on demand from a storable, non-degrading pool of high quality polynucleotide assembly blocks by a simple amplification process, eliminating the need for repeated assembly of oligos. Moreover, the use of balanced and standardized double-stranded polynucleotide assembly blocks as the basic unit for target polynucleotide assembly limits and/or prevents the propagation of sequence errors from the raw oligo pool into the final product, making the yield of perfect sequences, and therefore the cost per base, relatively independent of the length of the assembled polynucleotide.

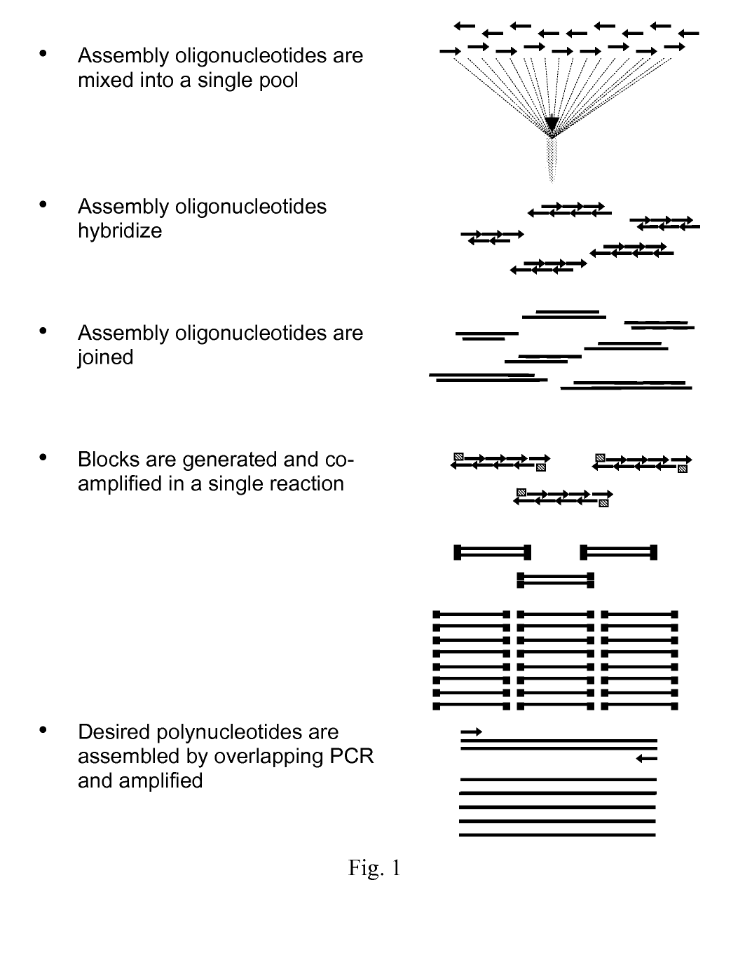

As described in the examples herein, successful polynucleotide assembly has been demonstrated from complex pools containing a thousand or more distinct oligonucleotides in sub-femtomolar amounts, and results in polynucleotides of interest with error rates significantly less than what is possible using current methods. An illustrative example of the assembly of assembly oligonucleotides into polynucleotide assembly blocks and polynucleotide assembly blocks into a target polynucleotide is shown schematically in FIG. 1.

### Assembly Oligonucleotide and Assembly Oligonucleotide Pool Composition

In various aspects of the methods, compositions, and kits disclosed herein, an assembly oligonucleotide pool is provided, comprising a plurality of assembly oligonucleotides from which one or more polynucleotide assembly blocks can be assembled. (It will be apparent to persons having ordinary skill in the art that the same methods can be used to assemble one or more target polynucleotides directly from assembly oligonucleotides, particularly if the target polynucleotides are shorter than about 500, 450, 400, 350, 300, or 250 base pairs in length, and such use of the methods, compositions, and kits disclosed herein are within the scope of the invention, although assembly of polynucleotide assembly blocks is preferred for most applications.)

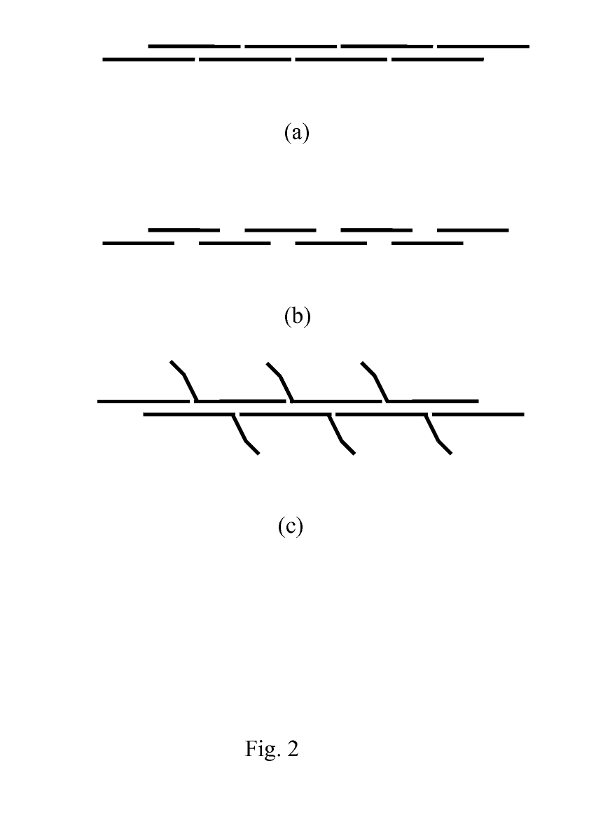

The assembly oligonucleotide pool comprises assembly oligonucleotides whose overlap segments are mutually reverse complementary and therefore capable of hybridization one to another when incubated under appropriate conditions and temperature. Each internal assembly oligonucleotide hybridizes to two other assembly oligonucleotides that each correspond to the opposite sense strand, and each terminal assembly oligonucleotide hybridizes to one internal assembly oligonucleotide corresponding to the opposite sense strand. When all of the assembly oligonucleotides corresponding to a single polynucleotide assembly block have hybridized, they form a single hybridized complex spanning the entire sequence of the polynucleotide assembly block. If the assembly oligonucleotides are fully overlapping, the resulting hybridized complex spans the entire sequence of both strands, without gaps, as illustrated schematically in FIG. 2(a). If the assembly oligonucleotides are partially overlapping, single-stranded gaps may exist in the resulting complex, as illustrated in FIG. 2(b). One or more assembly oligonucleotides may also overlap the adjacent assembly oligonucleotide of the same sense strand, resulting in redundant overlaps in all or part of the sequence of the hybridized complex, as illustrated in FIG. 2(c). Whether one or more assembly oligonucleotides corresponding to a polynucleotide assembly block is fully overlapping, partially overlapping, or redundantly overlapping, the resulting hybridized complex may further comprise 3′ and/or 5′ overhangs at either or both termini or may comprise blunt ends at either or both termini. To the extent that partially overlapping assembly oligonucleotides are used, resulting in single-stranded gaps in the hybridized complex, the gaps can be filled to complete the polynucleotide assembly block, which may be accomplished by PCR synthesis of the missing subsequence(s) (in which case any errors in the gap region(s) will be propagated), and/or by hybridizing additional oligonucleotides that are reverse complementary to the gap region(s).

In various embodiments where one or more target polynucleotides is to be assembled from more than a single polynucleotide assembly block, the two or more polynucleotide assembly blocks are joined to obtain the desired target polynucleotide. This may be accomplished by any method operable to join two polynucleotide assembly blocks. In various embodiments, target polynucleotides are assembled by joining two or more polynucleotide assembly blocks using overlap extension PCR, wherein polynucleotide assembly blocks to be joined have a region of common sequence, typically at or near opposite termini, so that upon melting and reannealing each primes the other, allowing extension of each chain using the other as template, after which the joined duplex may be amplified, all as described in greater detail infra. Any other method operable for joining polynucleotide assembly blocks may also be used, including without limitation and by way of example only, blunt-end ligation or sticky-end ligation. The method used can be considered in determining the appropriate sequence of the polynucleotide assembly blocks at the region of joining; if the overlap PCR approach is to be used, the sequences of the polynucleotide assembly blocks, and consequently the sequences of the assembly oligonucleotides from which the polynucleotide assembly blocks are assembled, can be designed to include the overlaps. If it is desired for polynucleotide assembly blocks to comprise adaptors, the sequences of the polynucleotide assembly blocks, and consequently the sequences of the assembly oligonucleotides corresponding to the adaptor region(s), can be designed to include the overlaps. Other desired sequence elements may also be included in the sequences of the polynucleotide assembly blocks, including without limitation and by way of example only, one or more primers for use in selectively amplifying one or more particular polynucleotide assembly blocks or groups of polynucleotide assembly blocks from a polynucleotide assembly block pool.

### Overlap Segment Design

In one embodiment, the methods, compositions, and kits of the invention provide assembly oligonucleotides that are designed to foster the preferential assembly of assembly oligonucleotides whose sequence is exactly correct and prevent or avoid the hybridization of correct assembly oligonucleotides with oligonucleotides whose sequence is incorrect. Any composition(s) and/or method(s) that is/are operable to produce hybridization of overlap segments in such a way that hybridization of overlap segments having the correct sequence is favored over other possible hybridizations can be used in accordance with the present invention. Without limiting the generality of the foregoing, in various embodiments, hybridizations of assembly oligonucleotides is performed at a temperature at which hybridization of pairs of overlap segments that are exactly reverse complementary are able to occur more readily than hybridizations involving incorrect sequences. The ability to select against incorrect hybridizations can be improved to the extent that correct hybridizations of the overlap segments of the assembly oligonucleotides anneal at a temperature distinctly higher than the temperature at which hybridizations with incorrect oligonucleotides anneal, so that it is possible to segregate correctly hybridized duplexes from incorrect hybridizations by setting the temperature used for hybridization during assembly of assembly oligonucleotides at a temperature that is low enough to allow the correct hybridizations to form but high enough to prevent incorrect hybridizations from forming.

It is well known to persons having ordinary skill in the art that as nucleic acids are heated under appropriate conditions, melting takes place over a range of temperatures, so that at the nominal melting temperature of a given sequence, half of the duplexes of that sequence with its reverse complement will be melted and half will be annealed. As the temperature is raised or lowered above and below the nominal melting temperature, varying proportions of the hybridized duplexes will remain annealed. This phenomenon is described by the melting relation

$$θ = \frac{1}{{e}^{\frac{Δ H}{RT} + \frac{Δ S + Rln ( \frac{C}{2} )}{R}}}$$

where θ is the percent of duplexes remaining annealed at a temperature of T in degrees Kelvin, C is the concentration of duplexes present if all were annealed, ΔH is the change of enthalpy and ΔS the change in entropy, respectively, on going from the annealed to the melted state, and R is the ideal gas constant. At a temperature within a few degrees Celsius of the melting temperature in either direction, not all of the duplexes will be melted or annealed; rather, the proportion of the duplexes given by θ in the foregoing equation will remain annealed.

The melting temperature of a given hybridized duplex will depend upon the length, the sequence, and the nature and position of any differences between the sequence of the first strand and the reverse complement of the second strand. The melting temperature for a given sequence with respect to its exact reverse complement may be estimated according to the formula:

$${T}_{m} = 64.9 + \frac{41 ( {N}_{G} + {N}_{C} - 16.4 )}{L}$$

where N_{G} is the number of G residues in the overlap segment, N_{C} is the number of C residues in the overlap segment, and L is the number of all residues in the overlap segment.

To the extent that the overlap segments of the assembly oligonucleotides in an assembly oligonucleotide pool differ in sequence in ways that affect their respective melting temperatures and melting curves, the result will be that, at a given temperature at which hybridization of assembly oligonucleotides is carried out, the various pairs of reverse complementary overlap segments will be melted in differing proportions. An assembly oligonucleotide pool may comprise, in addition to the correctly synthesized assembly oligonucleotides, a distribution of other oligonucleotides having sequences that differ from the correct sequences in length and/or composition. The extent to which these will introduce error by hybridizing with correct assembly oligonucleotides at the temperature used for assembly of the assembly oligonucleotides will depend upon the specific incorrect sequences present and the concentrations at which they are present. In principle, if the exact sequences and concentrations of all oligonucleotides present were known, it would be possible to make a close estimate of the relative quantities of various correct and incorrect hybridizations that would form at a given temperature, and thereby select a temperature that optimally excludes the unwanted hybridizations from forming. As a practical matter, the sequences and concentrations of the incorrect oligonucleotides will not usually be known or determinable, so it may be useful to base the temperature design of the overlap segments upon mismatch-exclusion temperature characteristics reflecting reasonable estimates or heuristics.

In various embodiments of the invention, the melting temperatures of all overlap segments of all assembly oligonucleotides present in the assembly oligonucleotide pool are within a range not exceeding about 10, 9, 8, 7, 6, 5, 4, 3, 2, or 1 degree Celsius. In other embodiments, the melting temperatures of all overlap segments of all assembly oligonucleotides corresponding to the polynucleotide assembly blocks from which a target polynucleotide is to be assembled are within a range not exceeding about 10, 9, 8, 7, 6, 5, 4, 3, 2, or 1 degree Celsius. In other embodiments, the melting temperatures of all overlap segments of all assembly oligonucleotides corresponding to one or more polynucleotide assembly blocks are within a range not exceeding about 10, 9, 8, 7, 6, 5, 4, 3, 2, or 1 degree Celsius.

In other embodiments, the single-mismatch annealing temperature of an overlap segment is taken as conservatively representative of the population of incorrect sequences. Other factors being equal, sequences that differ from the exact reverse complement of an overlap segment at only a single position will, in general, hybridize with the overlap segment more readily and at a higher temperature than sequences that differ at more than one position. In various embodiments, the melting temperature of the overlap segment having the lowest melting temperature of all overlap segments of all assembly oligonucleotides present in the assembly oligonucleotide pool is at least about 10, 9, 8, 7, 6, 5, 4, 3, 2, or 1 degree Celsius higher than the single-mismatch annealing temperature of the overlap segment having the highest single-mismatch annealing temperature of all overlap segments of all assembly oligonucleotides present in the assembly oligonucleotide pool. In other embodiments, the melting temperature of the overlap segment having the lowest melting temperature of all overlap segments of all assembly oligonucleotides corresponding to the polynucleotide assembly blocks from which a target polynucleotide is to be assembled is at least about 10, 9, 8, 7, 6, 5, 4, 3, 2, or 1 degree Celsius higher than the single-mismatch annealing temperature of the overlap segment having the highest single-mismatch annealing temperature of all overlap segments of the same assembly oligonucleotides. In other embodiments, the melting temperature of the overlap segment having the lowest melting temperature of all overlap segments of all assembly oligonucleotides corresponding to one or more polynucleotide assembly blocks is at least about 10, 9, 8, 7, 6, 5, 4, 3, 2, or 1 degree Celsius higher than the single-mismatch annealing temperature of the overlap segment having the highest single-mismatch annealing temperature of all overlap segments of the same assembly oligonucleotides.

In various embodiments the codon frequencies of the correct assembly oligonucleotides in the assembly oligonucleotide pool, and consequently the codon frequencies of the polynucleotide assembly blocks and polynucleotide assembly block pool, reflect the recoding of sequences in accordance with the recoding methods disclosed herein. In some embodiments the assembly oligonucleotides, assembly oligonucleotide pool, polynucleotide assembly blocks, and/or polynucleotide assembly block pool comprise exclusively codons that are not low frequency codon types, and comprise at least one codon that is not a maximum frequency codon type, all with respect to the expression environment of interest in which the target polynucleotide is intended to be expressed. In some embodiments the distribution of codon frequencies for the assembly oligonucleotides, assembly oligonucleotide pool, polynucleotide assembly blocks, and/or polynucleotide assembly block pool is also, or alternatively, statistically distinct from the natural distribution of codon frequencies of the expression environment in which the target polynucleotide is to be expressed, as determined by a suitable statistical test for determining whether two frequency distributions are distinct, such as, by way of non-limiting example, a chi-squared test reflecting a probability of 90, 95, 96, 97, 98, 99, 99.5, or 99.9 percent probability that the two distributions are distinct.

### Recoding

In specifying an assembly oligonucleotide pool such that the overlap segments of the assembly oligonucleotides will have the desired temperature characteristics, while maintaining a desired encoding and other desired sequence characteristics, it may be useful to recode the source sequence, and/or subdivide the target sequence and its reverse complement into assembly oligonucleotides, in such a way as that sequences allocated to overlap segments conform to the desired melting temperature, single-mismatch melting temperature, and other criteria deemed important. Recoding and subdivision of the target sequence and its reverse complement into assembly oligonucleotides may be performed in either order or via an algorithm that recodes and subdivides in repetitively alternating steps or in an iterative manner.

In some embodiments and applications recoding for temperature characteristics is constrained by the need or desire to add, remove, preserve, diminish, or enhance other characteristics that are determined in whole or part by the sequence. Without limiting the generality of the foregoing, examples of potentially desirable recoding goals include:
1. Promoting balanced amplification. It is generally preferred that when polynucleotide assembly blocks are amplified following assembly, the amplification proceed in a balanced manner so that all polynucleotide assembly block species are amplified in approximately uniform quantities. Adjustment to obtain GC content uniformity, to the extent feasible, over the length of each polynucleotide assembly block may be useful for balanced amplification, by causing polynucleotide assembly blocks to melt and anneal at relatively uniform rates, and by fostering uniform movement of the polymerase along the template strand. Sequence adjustment to avoid undesired sequence features or motifs may also improve uniformity of amplification rates; by way of non-limiting example, sequence motifs tending to promote the formation of secondary structure in the single strands should preferably be adjusted where present, since secondary structure in the template strand may stall polymerase movement and interfere with amplification. Sequence features such as direct and inverted repeats more than about 7 nucleotides in length should preferably be avoided in the recoded sequence since these may tend to cause hairpin formation.
2. Providing for improved transcription of the target polynucleotide. In embodiments where all or part of the target polynucleotide is intended to be transcribed, it may be useful to adjust the sequence to enhance transcription and/or eliminate sequence features or motifs that interfere with transcription, such as its rate or processivity.
3. Providing for improved RNA stability. It may be useful to eliminate sequence features that would result in degradation signals in the transcript or otherwise adversely affect RNA stability.
4. Providing for efficient initiation of translation. In embodiments where all or part of the target sequence is intended to encode a desired polypeptide or protein, it may be useful to optimizing ribosome binding site sequence and position to the extent feasible, and to adjust the sequence to avoid the occurrence of cryptic initiation signals. It may also be desirable to adjust sequences to eliminate mRNA secondary structure, which may interfere with initiation of translation and/or with the efficiency of translation.
5. Providing for improved translation rate and processitvity. In embodiments where all or part of the target sequence is intended to encode a desired polypeptide or protein, recoding within the coding sequence is of course limited to substitutions of synonymous codon types that encode the same amino acid as the codon being replaced. In other embodiments, where all or part of the target sequence is required to conform to other constraints, the recoding is similarly limited to substitutions conforming to those constraints. It is known to persons having ordinary skill in the art that each organism has a characteristic codon preference; degenerate codon types appear in wild type protein-coding in relative frequencies that are in part determined mainly by the abundance of tRNA's corresponding to each of the various codon types. Where a target polynucleotide sequence is to be expressed in a particular expression environment, it is preferable either to conform the sequence to the codon types having the maximum frequencies in the expression environment chosen (so that only abundant tRNA's are called upon), or to conform the sequence to the distribution of frequencies characteristic of the expression environment (so that all available tRNA's are used approximately in proportion to their abundance).
6. Elimination of undesired restriction sites and/or introduction of desired restriction sites or other molecular recognition sequences. Particularly where target polynucleotides are intended to be cloned into expression vectors, incorporation of appropriate restriction sites at specific locations may be required. In applications where restriction enzymes will be used sequences should preferably be adjusted to ensure that unintended restriction sites recognizable by the enzymes used do not appear.

The foregoing enumeration is illustrative of some of the many purposes for which it may be desirable to recode sequences. It will be apparent to persons having ordinary skill in the art that there are many other possible motivations for sequence recoding, which may extend to any of the many considerations affecting amplification and/or expression. Optionally, particular subsequences in a target sequence may be masked against recoding so as to avoid modifying a desired restriction sequence, regulatory sequence, primer sequence, or other subsequence whose exact preservation is desired for any reason.

This embodiment of the methods, compositions, and kits of the invention offers a novel solution to the difficult challenge presented when there is a need to recode for improved temperature characteristics while at the same time providing for efficient expression in a given expression environment. The currently accepted methods for obtaining efficient expression—recoding to maximum frequency codon types for the expression environment, or recoding to conform to the codon usage frequencies of the expression environment (perhaps with the lowest frequency codons replaced)—precludes any recoding for temperature characteristics that facilitate segregation of exactly reverse complementary hybrids from hybridizations with mismatches, because every codon within any coding region is already specified as a result of the recoding for efficient expression, leaving no flexibility to recode within any coding region. The inventors' discovery that efficient expression requires only the elimination of the lowest frequency codon types (those representing less than about 12, 11, 10, 9, 8, 7, 6, or 5 percent of the codon types coding for the same amino acid in the expression environment in which expression is desired), and that once the lowest frequency codons are replaced, recoding to the highest frequency codon types or recoding to conform to usage frequencies does not materially improve results further, opens scope for additional recoding for other purposes, and enables the novel recoding method disclosed herein. By way of non-limiting example, for optimal mammalian gene expression only 8 codons need to be avoided: GCG (Ala), CGA (Arg), CGT (Arg), CTA (Leu), TTA (Leu), CCG (Pro), TCG (Ser), ACG (Thr). For recoding a gene for dual use in either a mammalian or E. coli or such based systems such as in vitro translation lysates, only 11 codons need to be avoided: GCG, AGA, AGG, CGA, CGT, ATA, CTA, TTA, CCG, TCG, ACG.

In some embodiments where all or part of a target polynucleotide is intended for expression in an expression environment, recoding of the target polynucleotide sequence may be performed according to a method comprising the steps of:
1. Determining, with respect to the expression environment, the low frequency codon types, the maximum frequency codon types, and the mid-frequency codon types;
2. Replacing all low-frequency codon types occurring within any coding regions of the sequence with mid-frequency or maximum-frequency codon types;
3. Making one or more acceptable substitutions that result in improvement of at least one characteristic of the sequence.
   Steps 2 and 3 may be performed in either order, or may be performed iteratively by alternating between the two and making fewer than all desired replacements and/or substitutions at each iteration, continuing to iterate until all desired replacements and substitutions have been made. Determination of the low, mid-, and maximum frequency codon types may be made by categorizing the codon types encoding each amino acid according to their frequencies in the expression environment of interest, where frequencies are expressed as the frequency of the codon type in question as a percentage of the frequencies of all codons encoding the same amino acid in the expression environment of interest. The codon frequencies may be determined by any method known to persons having ordinary skill in the art, including without limitation and by way of example only, by counting the frequencies of codons in wild type sequences corresponding to the expression environment of interest, by experimental evaluation of tRNA abundance, or by consulting references or databases in which codon usage frequencies are published. The maximum frequency codon types are those codon types that, in the expression environment of interest, have frequencies greater than or equal to the frequencies of all other codon types that encode the same amino acid. The low frequency codon types are those codon types that, in the expression environment of interest, have frequencies below a minimum efficiency threshold. The minimum efficiency threshold should preferably be a frequency chosen such that a sequence comprising codon types all of which have a frequency greater than the minimum efficiency threshold will be expressed with reasonable efficiency in the expression environment of interest. The minimum efficiency threshold may be based on experiment evaluating the effect of the presence of various codon types on expression efficiency, or may be set at a level found heuristically to give acceptable results in the same or other expression environments, such as about 12, 11, 10, 9, 8, 7, 6, or 5 percent. The mid frequency codon types are those codon types that are neither low frequency codon types nor maximum frequency codon types.

Replacement of all low frequency codon types in any coding regions is sufficient to ensure that translation will not be made inefficient due to insufficient abundance of tRNA species corresponding to the codons used. The sequence may then be further recoded to accomplish any other objectives by making acceptable substitutions. Within regions encoding polypeptides or proteins intended to be expressed, acceptable substitutions are any in-frame substitutions that replace a first codon with a second codon, where, the second codon encodes the same amino acid as the first codon and the second codon is not a low frequency codon. Within regions comprising sequence regions or motifs having other desired functions, such as, by way of non-limiting examples only, promoters, enhancers, splice sites, ribosome binding sites, or localization signals, acceptable substitutions are any substitutions of one or more nucleotides for one or more other nucleotides where the substitution preserves the desired function of the sequence region or motif at a level adequate for the intended application, and does not unacceptably impair the function of other regions of the target polynucleotide (such as by causing an unwanted frame shift or by introducing an unwanted restriction site or other motif). Within sequence regions not encoding a product to be expressed, and not having any other function required for the intended application (such as, by way of non-limiting example, intron regions not important for regulation or splicing, and regions whose only function is to act as a spacer or linker between other sequences), acceptable substitutions are any substitution of any one or more nucleotides for any other one or more nucleotides. Acceptable substitutions in regions not encoding a polypeptide or protein to be expressed may also include insertions or deletions of one or more nucleotides, provided that the insertion or deletion does not cause a frame shift in a coding region, or otherwise impair a function required for the intended application.

Choice of the acceptable substitutions to be made may be made by any method operable for selecting substitutions that improve the characteristic of the sequence sought to be improved. Where it is known or can be determined what effect a given substitution will have or is likely to have with respect to the characteristic sought to be improved, substitutions tending to have the desired effect can be chosen. Where the characteristic sought to be improved is one as to which the effect of a given substitution is not known or determinable, or where it is sought to optimize two or more characteristics that may be affected differently by substitutions, suitable substitutions may be chosen by iteratively making one or more tentative substitutions, evaluating the resulting sequence with respect to the characteristic of interest, and accepting the one or more substitutions based on if the characteristic was improved and rejecting the one or more substitutions otherwise.

The characteristic sought to be improved may be any characteristic affected by the identities and positions of nucleotide species in the sequence. In some embodiments, a goal of recoding is to improve the mismatch-exclusion temperature characteristics of a sequence or part thereof.

### Algorithm and Method for Oligo Design

Any method or algorithm may be used for specifying the sequences of assembly oligonucleotides that is operable for deriving, from one or more target polynucleotide sequences, the sequences of assembly oligonucleotides of the desired sizes or size distribution, having overlap segments whose temperature characteristics allow preferential assembly of correct sequences, and which, when joined by hybridization of the overlaps and filling of any gaps, together make up the sequence of polynucleotide assembly blocks that comprise any desired overlaps with adjacent blocks, adaptors, primers, or other desired sequence elements.

In some embodiments, the methods, compositions, and kits disclosed herein provide for one or a plurality of polynucleotide assembly block pools from which a plurality of target polynucleotides can be selectively assembled from polynucleotide assembly blocks and amplified. The plurality of target polynucleotides may, by way of non-limiting example, comprise the genes present in an organism, a chromosome of an organism, or other subset of the genome of an organism. In such embodiments the method for specifying the sequences of the assembly oligonucleotides may comprise additional steps for allocating the target polynucleotides to blocks and/or to polynucleotide assembly block pools. An exemplary embodiment of the method for specifying the sequences of assembly oligonucleotides corresponding to a plurality of target polynucleotides and to be synthesized on a plurality of arrays, with the assembly oligonucleotides corresponding to each array corresponding to a single polynucleotide assembly block pool, comprises the steps of at least 1, 3, and 5 below, and may optionally comprise the other recited steps:
1. Recoding the target polynucleotide sequences;
2. Allocating target polynucleotide sequences to polynucleotide assembly block pools;
3. Allocating target polynucleotide sequences to polynucleotide assembly blocks;
4. Determining the position of the boundaries of target polynucleotide sequences within polynucleotide assembly blocks;
5. Determining the boundaries of overlap segments in the target polynucleotide sequence or part thereof and its reverse complement; and
6. Determining the boundaries of assembly oligonucleotides.

In some embodiments the method further comprises inserting desired adaptor sequences prior to or in connection with determining the boundaries of assembly oligonucleotides. The steps may be performed in any order. Two or more steps may be combined in a single step. Any one or more steps may be performed iteratively. Any step may be performed with respect to multiple sequences, single sequences, or one or more parts of sequences before a different step is begun.

In embodiments where the number of distinct target polynucleotide sequences is larger than the number that can be synthesized on a single array or otherwise conveniently provided in a single assembly oligonucleotide pool, a plurality of assembly oligonucleotide pools can be used, and target polynucleotide sequences can be allocated to particular assembly oligonucleotide pools. Such allocation can be made in such a way that the assembly oligonucleotides required to assemble all polynucleotide assembly blocks required for assembly and amplification of a target polynucleotide are present in the same assembly oligonucleotide pool. In making the allocation it may be useful to take into account the mismatch-excluding temperature characteristics of the assembly oligonucleotides assigned to each assembly oligonucleotide pool, so as to improve the exclusion of incorrect sequences from hybridization in each assembly oligonucleotide pool. By way of non-limiting example, it may be useful to allocate target polynucleotides to assembly oligonucleotide pools in such a way as to make the overlap segment melting temperatures as uniform as possible within each pool, and/or to maximize the difference between the melting temperature of the overlap segment having the lowest melting temperature and the single-mismatch annealing temperature of the overlap segment having the highest single-mismatch annealing temperature, within each assembly oligonucleotide pool. Where the number of distinct target polynucleotide sequences is small enough to allow assembly of all the target polynucleotide sequences from assembly oligonucleotides that can be synthesized on a single array or otherwise obtained in a single assembly oligonucleotide pool, the step of allocating target polynucleotide sequences to polynucleotide assembly block pools may be omitted, although in such embodiments division into multiple assembly oligonucleotide pools may still be beneficial for other purposes, such as, by way of non-limiting example, grouping assembly oligonucleotides into assembly oligonucleotide pools in such a way as to improve the distribution of mismatch-exclusion temperature characteristics in each pool.

In allocating target polynucleotides to polynucleotide assembly blocks and determining boundaries, it may be desirable to provide for target polynucleotide sequences to abut each other within polynucleotide assembly blocks so that assembly oligonucleotide sequence is not wasted providing for unused “spacer” sequence between target polynucleotides. It is not necessary for target polynucleotides to abut each other in the same order that they appear in the genome of an organism or in any other particular order, and the order in which target polynucleotides abut each other may be rearranged to accommodate polynucleotide assembly block layout. In some preferred embodiments target polynucleotides are ordered in such a way as to place the boundaries between abutting target polynucleotides at loci at least 10, 20, 30, 40, 50 or more nucleotides distant from either polynucleotide assembly block terminus, so as to avoid the need for joining polynucleotide assembly blocks merely to obtain the last few nucleotides of a target polynucleotide sequence during joining and amplification.

In some embodiments, recoding of the target polynucleotide sequence may be performed according to a recoding algorithm as disclosed herein, wherein low frequency codon types are replaced where they occur within coding regions and the sequence is also recoded to improve one or more mismatch-exclusion temperature characteristics; overlap segments may be determined by subdividing the target polynucleotide sequence and its reverse complement into contiguous overlap segments having, individually or as a set, desired mismatch-exclusion temperature characteristics; and the boundaries of the assembly oligonucleotides may be determined by combining adjacent pairs of overlap segments to specify the sequences of internal assembly oligonucleotides and combining single overlap segments with adaptor sequences to specify the sequences of terminal assembly oligonucleotides, on both strands in such a way that the boundaries of the assembly oligonucleotides corresponding to one strand are staggered or displaced relative to the assembly oligonucleotides corresponding to the other strand.

An illustrative embodiment of an algorithm and method for determining the sequences of the assembly oligonucleotides making up the assembly oligonucleotide pool is described in detail in Example 1. It will be apparent to persons having ordinary skill in the art that many variations of the method and algorithm are possible, and will produce equivalent results, and the disclosure hereof extends to all such variations and equivalents. Without limiting the generality of the foregoing, and by way of example only, the recoding of the sequence and the subdivision of the sequence into overlap segments can be performed in any order, or as part of a single step, or by repetitive alternation and iterative improvement, or in any other way that is operable to specify finished assembly oligonucleotide sequences having the desired sequence and temperature characteristics; sequences may be segregated directly into assembly oligonucleotides without being first segregated into overlap segments; and the algorithm may be modified to allow specification of assembly oligonucleotides that are not fully overlapping, or that are redundantly overlapping, where desired. The specification of assembly oligonucleotides may be accomplished in whole or part using iterative optimization methods such as genetic or evolutionary algorithms or simulated annealing, wherein a starting population of candidate assembly oligonucleotides is iteratively modified until desired criteria have been met.

### Polynucleotide Assembly Block Composition

In a preferred embodiment, the methods of the invention are used to produce a polynucleotide assembly block pool comprising a plurality of polynucleotide assembly blocks, one or more of which can be selectively amplified from the pool in whole or part, and, where desired, joined to all or part of one or more other polynucleotide assembly blocks to produce longer polynucleotides. Polynucleotide assembly blocks comprise double-stranded polynucleotides, and may optionally further comprise single-stranded overhangs in either or both strands, which may be at either terminus. The polynucleotide assembly blocks corresponding to a single polynucleotide assembly block pool may be designed for unbiased simultaneous amplification by providing a removable adaptor for hybridization of a block amplification primer during amplification, by making the lengths of the polynucleotide assembly blocks relatively uniform, and by including as recoding objectives during assembly oligonucleotide design the improvement of other factors related to balanced amplification, including without limitation uniformity of GC content and avoidance of sequence features that may result in uneven polymerase movement.

Polynucleotide assembly blocks comprise a minimum length of 80, 90, 100, 110, 120, 130, 140, 150, 160, 170, 180, 190, 200, or more nucleotides. There is no theoretical maximum length; although it may be preferable in some embodiments for polynucleotide assembly blocks assembled directly from assembly oligonucleotides to have lengths capable of assembly from about 8, 10, 12, 14, or 16 assembly oligonucleotides so as to maintain high efficiency of assembly, it is also possible to assemble polynucleotide assembly blocks from other polynucleotide assembly blocks, so that any size polynucleotide assembly block can readily be produced via iterated assembly. An advantage of the use of polynucleotide assembly blocks as the fundamental unit for assembly of target polynucleotides is that because oligonucleotides containing errors are selectively excluded during the assembly of polynucleotide assembly blocks, polynucleotide assembly blocks are of high quality and accuracy, so that propagation of the high error content of the oligonucleotide pool is prevented. As a result, accuracy, and consequently cost per base, remain relatively constant with increasing size. In various embodiments, where eight assembly oligonucleotides each approximately 50 nucleotides in length are used to assemble the polynucleotide assembly blocks, polynucleotide assembly blocks have lengths of approximately 250 nucleotides, including adaptors. In other embodiments, where ten assembly oligonucleotides each approximately 50 nucleotides in length are used to assemble the polynucleotide assembly blocks, polynucleotide assembly blocks have lengths of approximately 350 nucleotides, including adaptors.

Polynucleotide assembly blocks may be provided that have block overlaps with other polynucleotide assembly blocks so that two or more polynucleotide assembly blocks may be joined into a single polynucleotide by performing overlap PCR using primers corresponding to the termini of joined polynucleotides. The block overlaps are regions of identical sequence in adjacent polynucleotide assembly blocks. “Adjacent” polynucleotide assembly blocks are polynucleotide assembly blocks that are joined to form a polynucleotide of interest. The size and position within the sequence of the polynucleotide assembly block of any block overlap may be any size and/or position operable for block joining. Block overlaps of at least 4 nucleotides are sufficient to permit joining of adjacent polynucleotide assembly blocks; in various further embodiments, the block overlaps are at least 5, 6, 7, 8, 9, 10, 12, 14, 16, 18, 20, or more nucleotides. In a further embodiment, the block overlaps are at least as long as the assembly oligonucleotides on either side of the junction between adjacent polynucleotide assembly blocks. In a further embodiments, the block overlaps are as long as the shortest of the two polynucleotide assembly blocks that are overlapping. In a further embodiment, a block overlap is identical in length and sequence with an overlap segment of an assembly oligonucleotide from which the polynucleotide assembly block was assembled. In a preferred embodiment, all polynucleotide assembly blocks present in a single polynucleotide assembly block pool, having block overlaps for joinder of adjacent polynucleotide assembly blocks, have block overlaps whose lengths and temperature characteristics are approximately uniform so as to foster efficient and balanced multiplex amplification and joining. As will be apparent to those of skill in the art, the use of different size block overlaps may require different PCR conditions. Block overlaps may, but need not, comprise a terminus of a polynucleotide assembly block. In a non-limiting example, polynucleotide assembly blocks may comprise a block overlap and further comprise an adaptor sequence or other sequence positioned between the block overlap and the terminus of the polynucleotide assembly block. In various preferred embodiments, block overlaps are positioned adjacent to an adaptor sequence that comprises the terminus of the polynucleotide assembly block. A polynucleotide assembly block may comprise one block overlap, allowing it to be joined with one other polynucleotide assembly block, or may comprise two block overlaps, allowing both ends to be joined to other polynucleotide assembly blocks. In some embodiments the block overlaps within a single polynucleotide assembly block pool are of approximately uniform sizes.

Adaptor sequences can be added to polynucleotide assembly blocks to facilitate selective amplification of any desired one or more polynucleotide assembly blocks from a pool of assembled polynucleotide assembly blocks. The adaptor sequences can themselves be included, for example, in a terminal assembly oligonucleotide, or may be added to the desired region after block assembly using standard techniques in the art, including but not limited to ligation or PCR using primers complementary to a terminal region of a sequence to which adaptors are to be added, wherein the primers further comprise the adaptor sequence extending in the 5′ direction from the primer sequence. In one embodiment, unique 3′ adaptor sequences are added to each polynucleotide assembly block, permitting selective amplification of any polynucleotide assembly block from a pool of polynucleotide assembly blocks by use of appropriate adaptor primers, as well as also permitting selective amplification of all of the polynucleotide blocks for a polynucleotide of interest from a pool comprising polynucleotide assembly blocks for one or more polynucleotides of interest.

The adaptor sequences may optionally comprise recognition and cleavage loci for restriction enzymes to be used to cleave the adaptor sequences from the amplified polynucleotide assembly blocks. In one exemplary embodiment, the Mly I restriction enzyme is used to cleave the adaptor sequences from amplified polynucleotide assembly blocks. Mly I makes a double-stranded blunt ended cleavage at a locus 5 nucleotides from its recognition sequence, so when the Mly I recognition sequence is positioned in the 3′ adaptor sequence at 5 nucleotides from the boundary between the 3′ adaptor sequence and the remainder of the polynucleotide assembly block, the entire 3′ adaptor sequence is removed in a clean, double-stranded break. It will be apparent to a person having ordinary skill in the art that many other restriction enzymes can be used for this purpose; some choices of restriction enzymes and/or positions of restriction sequences in the 3′ adaptor sequence may result in 3′ or 5′ overhangs, and/or in retention of part of the 3′ retrieval sequence, but may nevertheless be practicable in the context of a particular application.

When restriction enzyme cleavage of adaptor sequences is intended, it is preferable to design the assembly oligonucleotides to insure that the restriction enzyme recognition sequence does not appear at positions other than the adaptor sequence. Furthermore, adaptor primers are preferably designed to minimize similarity between adaptor primer binding sequences and other sequences present in the polynucleotide assembly blocks or other sequences present in the relevant assembly oligonucleotide pool, to avoid inadvertent amplification of undesired sequences or of truncated sequences.

Method of Assembling Polynucleotide Assembly Blocks from Assembly Oligonucleotides

Assembly oligonucleotides may be assembled into larger polynucleotides by hybridizing two or more assembly oligonucleotides at their mutually reverse complementary overlap segments. Any method for assembling assembly oligonucleotides into intermediate fragments and intermediate fragments to produce one or more double stranded polynucleotide assembly blocks can be used, including but not limited to ligation chain reaction (LCR) and self-priming polymerase chain reaction (spPCR).

In embodiments where fully overlapping assembly oligonucleotides are used, assembly oligonucleotides can be hybridized and ligated together by ligase chain reaction, which may be performed according to any protocol operable for hybridizing and ligating fully overlapping single-stranded nucleic acids, or may be assembled by spPCR, or by combination of the above methods. In embodiments where partially or redundantly overlapping assembly oligonucleotides are used, polynucleotide assembly blocks may be assembled by overlap PCR. Upon incubation of assembly oligonucleotides under the chosen hybridizing conditions, intermediate fragments may form, comprising two or more assembly oligonucleotides but less than the number of assembly oligonucleotides sufficient to assemble an entire polynucleotide assembly block; these may be further amplified and assembled in additional cycles of melting and annealing so as to assemble complete polynucleotide assembly blocks.

In one non-limiting embodiment, hybridization of fully overlapping assembly oligonucleotides to form intermediate fragments, and assembly of intermediate fragments into a polynucleotide assembly block, can be accomplished in a single reaction mixture by ligase chain reaction (LCR). Any LCR protocol operable for assembling and ligating assembly oligonucleotides of the length, temperature characteristics, and composition present in the assembly oligonucleotide pool may be used. Without limiting the generality of the foregoing, and by way of example only, in one embodiment, LCR assembly may comprise performing hybridization/LCR ligation on an assembly oligonucleotide pool using exemplary conditions: 95° C. for 2 minutes, followed by 40 cycles 55° C. for 5 minutes, and 75° C. for 1 minute, wherein assembly oligonucleotides are phosphorylated and present at 50 pM concentration, in 1× Taq ligase buffer with 20u Taq ligase, optionally supplemented with 20% PEG in total volume 10 μl. It will be apparent to persons having ordinary skill in the art that other cycle times and temperatures, concentrations, volumes, buffer compositions, and enzymes may be used.

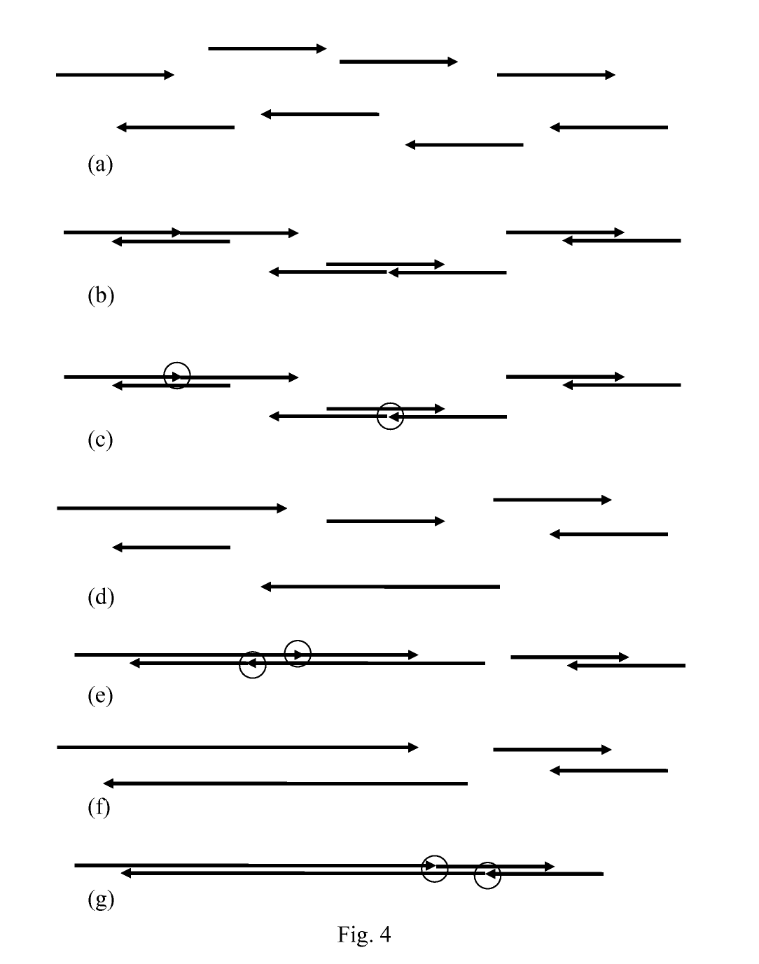

LCR assembly results in the hybridization and ligation of a plurality of overlapping assembly oligonucleotides into intermediate fragments and intermediate fragments into complete polynucleotide assembly blocks, as illustrated schematically in FIG. 4. The assembly oligonucleotides corresponding to a polynucleotide assembly block are initially present in the assembly oligonucleotide pool and the temperature is raised to ensure that all duplexes are melted (FIG. 4(a)). In the first cycle of annealing, assembly oligonucleotides associated randomly into duplexes (FIG. 4(b)) and abutting ends are ligated (FIG. 4(c), circles). In the next cycle of melting the duplexes dissociate (FIG. 4(d)), and on reannealing duplexes again form (FIG. 4(e)) and are ligated (FIG. 4(e), circles). In another cycle of melting (FIG. 4(f)) and annealing and ligating (FIG. 4(g)) the complete polynucleotide assembly block is formed. The use of stringent hybridization conditions and a hybridization temperature determined in accordance with the disclosures hereof favors hybridization of adjacent assembly oligonucleotides when no mismatches are present, and disfavors hybridization when mismatches are present. For LCR, the ligase used is unable to ligate two assembly oligonucleotides unless they are hybridized to a complementary sequence in a manner that exactly aligns the ends to be conjoined so that the termini of adjacent assembly oligonucleotides abut; thus, any oligonucleotides having errors in their sequences will be selected against for incorporation in the intermediate fragment, polynucleotide assembly block, or other ligated product. This helps ensure that a high proportion of the polynucleotide assembly blocks will be error-free, even when assembled from an oligo pool mixture containing many oligonucleotides having erroneous sequences. These methods can be used with an assembly oligonucleotide pool to produce a plurality of polynucleotide assembly blocks in a single reaction mixture. The result of LCR assembly in this embodiment is a polynucleotide assembly block pool, comprising a plurality of polynucleotide assembly blocks.

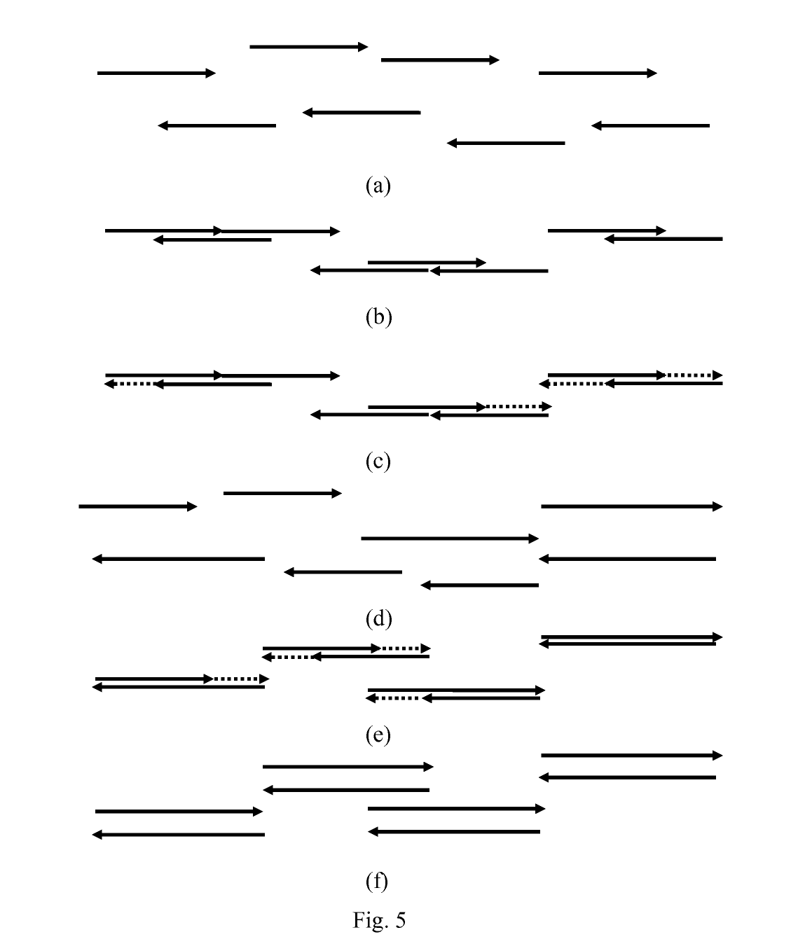

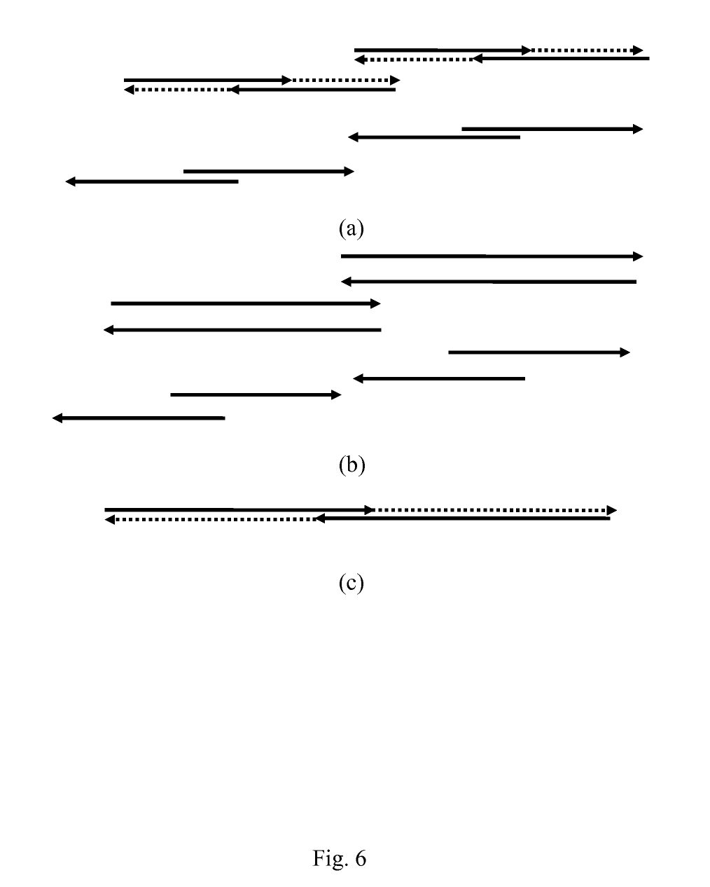

In another non-limiting embodiment, assembling polynucleotide assembly blocks comprises using spPCR, in which chain extension takes place using the single strand regions of hybridized overlap segments as templates and the hybridized complementary overlap segment as primer, with successive amplification steps assembling increasingly larger polynucleotides until each full polynucleotide assembly block is assembled. Polynucleotide assembly block assembly by spPCR is illustrated schematically in FIGS. 5 and 6. The assembly oligonucleotides corresponding to a polynucleotide assembly block are initially present in the assembly oligonucleotide pool and the temperature is raised to ensure that all duplexes are melted (FIG. 5(a)). In the first cycle of annealing, assembly oligonucleotides associated randomly into duplexes (FIG. 5(b) and overlapping 3′ ends are extended (FIG. 5(c). Duplexes are melted (FIG. 5(d)), then new duplexes are allowed to form and overlapping 3′ ends again extended (FIG. 5(e)). An additional cycle of melting (FIG. 5(f)) and annealing and extending (FIG. 6(a)) results in yet larger intermediate fragments. The duplexes are again melted (FIG. 6(b)) and single stranded fragments annealed and the 3′ overlaps extended, producing a complete polynucleotide assembly block (FIG. 6(c)). As with LCR assembly increasing the stringency of the annealing conditions by maintaining annealing temperature at 1, 2, 3, 4, 5, 6 degrees above the calculated Tm generally results in a lower expected probability of hybridization of mismatches resulting from any errors in overlap segment sequences, and is expected to lead to a higher proportion of error-free polynucleotide assembly blocks in the assembled polynucleotide assembly block pool.

It will be apparent to persons having ordinary skill in the art that various combinations of LCR and PCR assembly are possible, and that, whether assembly is performed by LCR, PCR, or a combination of the two, assembly oligonucleotides will hybridize in many possible combinations forming many species of intermediate fragments, and that the number of cycles required to assemble complete polynucleotide assembly blocks will vary. Use of spPCR for assembly of polynucleotide assembly blocks is preferred when adjacent sense or (antisense) assembly oligonucleotides are or can be designed to have gaps between them when hybridized to the complementary antisense (or sense) assembly oligonucleotides. Techniques and specific conditions for spPCR are known to those of skill in the art, and involve performing PCR on the pool of assembly oligonucleotides with overlap segments, where the assembly oligonucleotides alternate between sense and antisense directions, and the overlap segments serve to order the PCR fragments so that they selectively produce the polynucleotide assembly blocks. Without limiting the generality of the foregoing, and by way of example only, in one embodiment, PCR assembly may comprise performing spPCR on an assembly oligonucleotide pool using exemplary conditions: 95° C. for 1 min, followed by 40 cycles 95° C. for 10 sec, 58° C. for 20 sec, and 72° C. for 15 sec, wherein assembly oligonucleotides present at 50 pM concentration, in 1× iProof DNA polymerase buffer supplemented with 200 uM dNTP and 2 units of enzyme in total volume 100 μl.

In various preferred embodiments, the incubation of assembly oligonucleotides for hybridization is performed at a temperature that fosters exclusion of incorrect sequences from hybridizing with the overlap segments of assembly oligonucleotides. The temperature may be any temperature that permits hybridization of the desired assembly oligonucleotides into at least a minimum amplifiable quantity of assembled product, and favors hybridization of overlap segments with their exact reverse complements over hybridizations with incorrect sequences. In some embodiments where the range of the melting temperatures of the assembly oligonucleotides present in the assembly oligonucleotide pool is limited to 1, 2, 3, 4, or 5 degrees Celsius above or below a median, the temperature of incubation for hybridization is about 1, 2, or 3 degrees Celsius above the median melting temperature of the assembly oligonucleotides. In other embodiments, the temperature of incubation for hybridization is about 1, 2, 3, 4, or 5 degrees Celsius above the midpoint between the lowest melting temperature of any overlap segment of any assembly oligonucleotide in the assembly oligonucleotide pool and the highest single-mismatch annealing temperature of any such overlap segment.

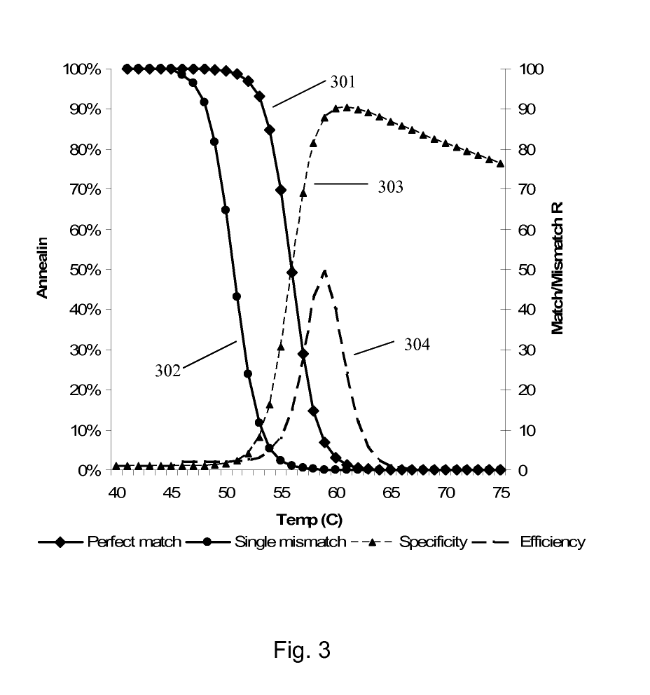

In other embodiments, the temperature of incubation for hybridization is the temperature that approximately maximizes the estimated ratio of the quantity of hybridized duplexes of overlap segments with their exact reverse complements to the quantity of hybridized duplexes of the same overlap segments with sequences that differ from the exact reverse complement at exactly one nucleotide position. This ratio may be estimated by (1) estimating the percentage of overlap segments hybridized with their exact reverse complements as a function of temperature, assuming equimolar concentrations of each pair of reverse complementary overlap segments, (2) estimating the percentage of overlap segments hybridized with single mismatches as a function of temperature, assuming equimolar concentrations of each pair of overlap segment and single mismatch, and (3) selecting a temperature at which the ratio of the former percentage to the latter is maximized. This method of estimation is illustrated graphically in FIG. 3, showing an illustrative melting curve 301 for overlap segments hybridizing with their exact reverse complements, an illustrative melting curve 302 for overlap segments hybridizing with sequences differing from the reverse complement by a single mismatch, the ratio 303 of the former to the latter, and the ratio heuristically adjusted 304 to disfavor temperatures at which both the numerator and denominator of the ratio are near zero. The melting curves shown in FIG. 3 reflect the melting relation previously described, in which the values of ΔH and ΔS are estimated for purposes of this illustration at −186,890 cal/M and −507 cal/K per M, respectively, for the hybridization of overlap segments with their exact reverse complements, and −183,890 cal/M and −507 cal/K per M, respectively, for the hybridization of overlap segments with single mismatches. It will be apparent to persons having ordinary skill in the art that there are many possible ways in which the estimate may be refined to the extent information is available concerning the actual erroneous sequences present and their respective concentrations relative to the correct assembly oligonucleotides. However, an incubation temperature for hybridization estimated in the manner described will give good results when used in conjunction with the methods disclosed herein.

In a preferred embodiment, the Tm of the overlap segments is between 50° C. and 74° C., and assembly is performed under stringent conditions to minimize hybridization of overlap segments with incorrect sequences. It will be apparent to a person having ordinary skill in the art that the stringency of nucleic acid hybridization conditions can be affected or adjusted by other factors, including without limitation and by way of example only, the choice of ligase or polymerase used in, for example, an LCR or PCR reaction, and the magnesium and other concentration and other composition of the ligase or polymerase buffer. Incubation for hybridization of assembly oligonucleotides is done under hybridizing conditions so as to result in hybridization of the overlap segments providing a plurality of double stranded intermediate fragments. The determination of appropriate hybridization conditions in light of the teachings herein is well known to those of skill in the art and will be based, at least in part, on the specific composition, sequence, distribution, and quantities of assembly oligonucleotide species present, and their overlap segments and overlap segment temperature characteristics.

### Polynucleotide Assembly Block Amplification.

The methods disclosed herein can be applied to an assembly oligonucleotide pool to produce a plurality of polynucleotide assembly blocks in a single reaction mixture. In various embodiments, the result of LCR or PCR assembly is a polynucleotide assembly block amplification pool, comprising at least 1, 10, 20, 40, 60, 80, 120, 140, 160, 180, 200, 220, 240, 260, 280, or 300 or more polynucleotide assembly blocks, which can be standardized for efficient and unbiased parallel amplification to allow all the polynucleotide assembly blocks present in the polynucleotide assembly block pool to be amplified together in a single reaction mixture. Prior to amplification the polynucleotide assembly blocks are at least partially double-stranded but may have single-stranded overhangs.

In some embodiments, after assembly of polynucleotide assembly blocks, the polynucleotide assembly block pool is purified to reduce the quantity of polynucleotide assembly blocks present whose sequences are not exactly correct, which may be accomplished by a number of methods such as a denaturation/renaturation step. This would cause strands to resort and a strand carrying any particular deletion would probabilistically re-anneal with a strand carrying a correct sequence at that position. This would a create loop-out spanning a deletion, which would provide a tag for cleavage with such enzymes as Mut S, or other type of modification, of the incorrect blocks. Size-exclusion, electrophoresis, or any other of the relevant purification methods known to persons having ordinary skill in the art could be used to separate the perfect from imperfect blocks.

In various embodiments, such as those where polynucleotide assembly blocks are assembled from array-synthesized assembly oligonucleotides, the assembly oligonucleotide concentrations are as low as femtomolar or attomolar, resulting in similarly low concentrations of assembled polynucleotide assembly blocks. The polynucleotide assembly block amplification pool can be amplified in whole or part after assembly of the polynucleotide assembly blocks, by any of the nucleic acid amplification techniques known to persons having ordinary skill in the art. Without limiting the generality of the foregoing, and by way of example only, polynucleotide assembly blocks may be amplified by PCR using primers complementary to block terminal regions. In various preferred embodiments, polynucleotide assembly blocks have adaptor sequences at or near their termini to facilitate amplification. In a further embodiment, all polynucleotide assembly blocks in a polynucleotide assembly block pool have “universal” adaptor sequences to facilitate simultaneous amplification of all polynucleotide assembly blocks, and polynucleotide assembly blocks are of relatively uniform length, temperature characteristics, and other characteristics relevant to efficiency of amplification, so as to foster balanced, unbiased amplification. Adaptors may optionally comprise restriction sites to enable their removal by restriction enzyme cleavage following amplification. In some embodiments, adaptors may be retained following amplification (facilitating further amplification of the entire block pool should additional quantities be desired), and are removed by the exonuclease activity of the polymerase used during joining and amplification of target polynucleotides as disclosed more fully below.

In one embodiment, all or a plurality of the polynucleotide assembly blocks in the polynucleotide block assembly pool comprise unique adaptor sequences to permit selective block amplification from the pool using appropriate amplification primers; in this embodiment, the pool may comprise polynucleotide assembly blocks for a plurality (two or more) polynucleotides of interest. In another embodiment, all or a plurality of the polynucleotide assembly blocks in the polynucleotide block assembly pool comprise universal adaptor sequences, permitting generalized amplification of the polynucleotide assembly blocks from the pool using appropriate primers. Since the polynucleotide assembly blocks are double stranded, they are much more stable than single stranded oligonucleotides; thus, they can be provided in solution, dried, frozen, or on a substrate, as desired for a given purpose. Polynucleotide assembly block pools can be created that are specific for any given one or more polynucleotides of interest. In various non-limiting embodiments, the polynucleotide assembly block pools may comprise pools specific for individual chromosomes, individual chromosomal loci, specific gene types (receptors, transcription factors, cytokines, etc.), genomic DNA, or any other polynucleotides of interest.

Where universal adaptors are used for multiplex amplification of polynucleotide assembly blocks having mutual block overlaps, it is preferred that no pair of polynucleotide assembly blocks that share a block overlap have the same pair of adaptor primer sequences in both of the polynucleotide assembly blocks comprising the pair; otherwise, amplification of the short overlap segment may occur in preference to amplification of entire blocks. It is possible to ensure that adjacent polynucleotide assembly blocks that share an overlap will not have identical adaptor pairs by employing two distinct adaptor pairs in an alternating fashion, so that, for example, where polynucleotide assembly block 1 overlaps with polynucleotide assembly block 2, which overlaps with polynucleotide assembly block c1 and 3 and polynucleotide assembly block 3 overlaps with polynucleotide assembly blocks 2 and 4, and so forth, amplification of block overlaps in preference to complete polynucleotide assembly blocks can be avoided by employing one pair of amplification primers for odd blocks and a different pair of amplification primers for even blocks.

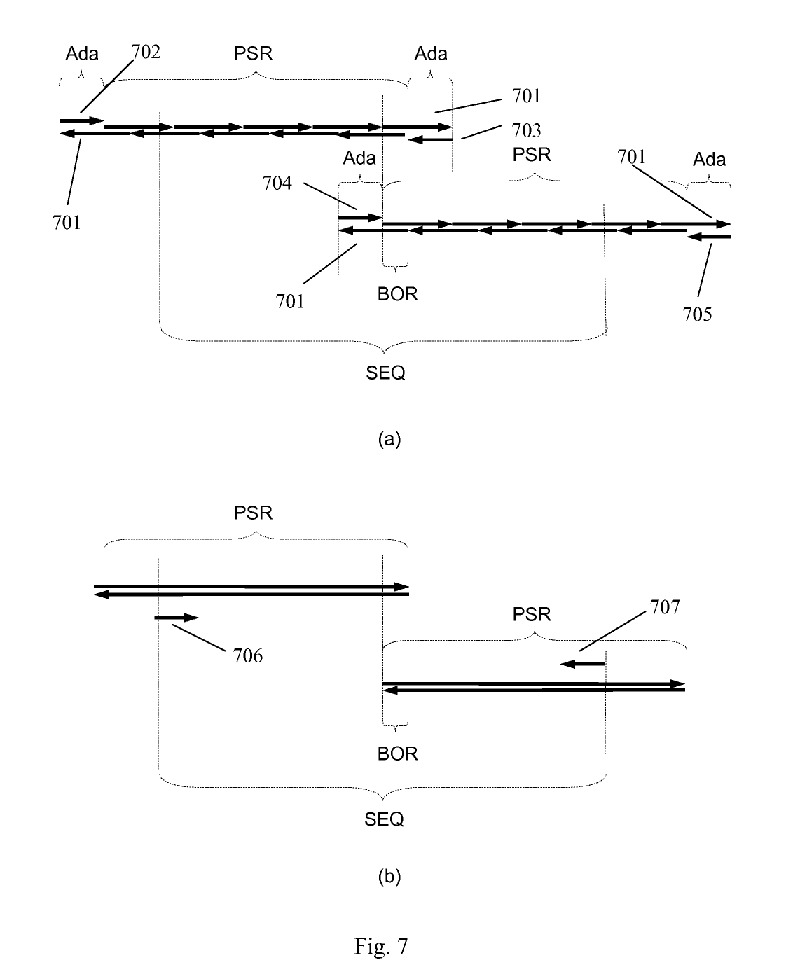

FIG. 7 illustrates schematically the composition of the polynucleotide assembly blocks and their amplification. As shown in FIG. 7(a), the polynucleotide assembly blocks are assembled from assembly oligonucleotides, with the terminal assembly oligonucleotides 701 comprising adaptor sequences ADA that comprise, respectively, sequences reverse complementary to forward 702 and reverse 703 primers for the first polynucleotide assembly block and forward 704 and reverse 705 primers for the second polynucleotide assembly block, with the primer pair for the second block being distinct from those for the first block so as to avoid amplification of the block overlap region BOR. The sequences of the two blocks are identical in the block overlap region BOR. The sequences of the first and second polynucleotide assembly blocks together span the sequence SEQ of the target polynucleotide. As shown in FIG. 7(b), after amplification of the polynucleotide assembly blocks and adaptor cleavage, two overlapping double-stranded polynucleotide assembly blocks are present, and can be joined and amplified using sequence specific primers 706, 707 corresponding to the termini of the desired sequence SEQ, as described more fully below.

In an exemplary embodiment, amplification of the polynucleotide assembly block amplification pool comprises performing PCR on a 5 μl aliquot of the multiplex polynucleotide assembly module mixture diluted 10 fold with water, to which has been added 1× iProof polymerase buffer, 200 uM dNTP, 500 nM retrieval primer, and 2u iProof polymerase, 40 cycles each at 95° C. for 10 seconds, 55° C. for 20 seconds, and 72° C. for 15 seconds. It will be apparent to persons having ordinary skill in the art that the volumes, buffer composition, polymerase, temperatures, and other parameters may vary depending upon the compositions of the polynucleotide assembly blocks being amplified, their temperature characteristics, and any other relevant factors.

### Polynucleotide Assembly Block Stock Pool

In a preferred embodiment, the polynucleotide assembly blocks are designed so that, after polynucleotide assembly block pool has been amplified, one or more target polynucleotides of interest can be selectively generated from the pool by amplifying and joining two or more polynucleotide assembly blocks using primers specific to the selected target polynucleotide(s). In this way a single polynucleotide assembly block stock pool can provide an unlimited supply of any desired target sequences represented in the pool. The polynucleotide assembly blocks in the polynucleotide assembly block stock pool are double-stranded and stable, and can readily be stored indefinitely so that the desired target polynucleotides can be obtained at will whenever desired. In some embodiments where the polynucleotide assembly blocks comprise adaptors for block amplification that are removable by restriction enzyme cleavage, the adaptors have been removed. In an exemplary embodiment, adaptor sequences including Mly I restriction recognition site are removed from the polynucleotide assembly blocks by Mly I restriction enzyme cleavage, in 50 μl comprising an aliquot of the amplified polynucleotide assembly block pool containing 50 ng of amplified polynucleotide assembly blocks, 1× Mly I restriction buffer with BSA, and 10u Mly I, incubated 2.5 hours at 37° C. In other embodiments, the adaptors used for polynucleotide assembly block amplification remain in place and are then removed during polynucleotide assembly block assembly and amplification of target polynucleotides via the exonuclease activity of the polymerase used for amplification.

### Block Assembly and Amplification of Polynucleotides of Interest

In various embodiments, the polynucleotide assembly block pool comprises a plurality of polynucleotide assembly blocks that comprise sequences that can be joined together to make up one or more target polynucleotides. A polynucleotide of interest may be obtained by joining and amplifying two or more polynucleotide assembly blocks by any procedure or method that is operable to produce a polynucleotide by joining and amplifying its sequence from two or more polynucleotide assembly blocks. Without limiting the generality of the foregoing, and by way of example only, in some embodiments the selective amplification of a polynucleotide of interest comprises performing PCR amplification using primers that are reverse complementary to the termini of the sequence to be joined and amplified, wherein the amplification further comprises overlap extension of overlapped strands from adjacent polynucleotide assembly blocks.

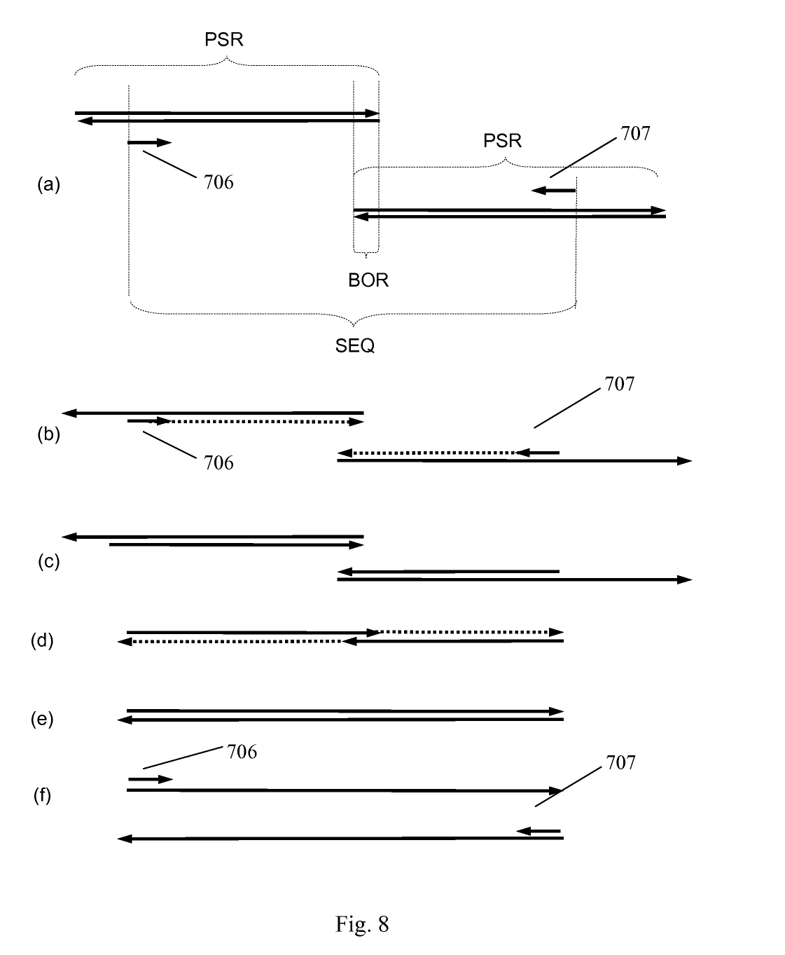

FIG. 8 illustrates schematically an embodiment of a method for joining and amplifying a target polynucleotide SEQ from two adjacent polynucleotide assembly blocks. As shown in FIG. 8(a), the two polynucleotide assembly blocks comprise block overlap regions BOR of identical sequence. As shown in FIGS. 8(b) and 8(c), upon melting and annealing of the sequence specific primers 706, 707, the polymerase extends from the primers, resulting in overlapping single strands of opposite sense, spanning the sequence SEQ of the target polynucleotide. Upon melting, followed by annealing of the overlapping strands (FIG. 8(d)), the polymerase extends from the overlap using the opposite sense strands as templates, resulting in a double-stranded polynucleotide having the desired sequence (FIG. 8(e)), which can then be PCR amplified (FIG. 8(f)) using the sequence specific primers 706, 707.

The method can be performed using any suitable PCR protocol. Without limiting the generality of the foregoing, in one non-limiting example, the amplification comprises performing PCR on a 15 μl aliquot comprising 2-2.5 ng of the polynucleotide assembly modules with adaptors removed, 1× iProof polymerase buffer, 200 uM dNTP, 0.6 μM each of the target sequence specific primers, and 0.6u iProof polymerase, in 40 cycles each at 95° C. for 10 seconds, 55° C. for 20 seconds, and 72° C. for 15 seconds. It will be apparent to persons having ordinary skill in the art that the volumes, buffer composition, polymerase, temperatures, and other parameters may vary depending upon the compositions of the polynucleotide assembly blocks being amplified, their temperature characteristics, and any other relevant factors.

It will be apparent to persons having ordinary skill in the art that, in principle, target polynucleotides of any length may be assembled from polynucleotide assembly blocks and selectively amplified in the manner disclosed herein, by joining two, three, four, five, or more adjacent polynucleotide assembly blocks having mutual overlaps. In some embodiments selective amplification of a longer target polynucleotide comprises an iterative process, wherein two or more polynucleotides are first obtained by joining and amplifying them from a plurality of polynucleotide assembly blocks, and a longer polynucleotide is then obtained by joining and amplifying the polynucleotides that were obtained in the preceding step. This iterative process may be carried out as many times as desired, assembling progressively longer polynucleotides with each iteration. In one non-limiting embodiment, amplifying a polynucleotide of interest from one or more polynucleotide assembly block comprises joining adjacent polynucleotide assembly blocks at regions of mutual overlap to form two or more polynucleotide contigs, and amplifying the polynucleotide of interest from the two or more polynucleotide contigs, wherein adjacent polynucleotide contigs are joined at regions of mutual overlap as necessary to produce the polynucleotide of interest. Contig assembly can be carried out, for example, by performing a self-priming PCR on the polynucleotide assembly block amplification pool, with or without removal of any adaptor sequences. This step is similar to and can be performed under the same conditions as described herein for spPCR-based polynucleotide block assembly from assembly oligonucleotides. Joining of the plurality of polynucleotide contigs can then be carried out as described herein for assembly of a polynucleotide of interest from a plurality of polynucleotide assembly blocks. This embodiment allows a user to compensate for any bias introduced by co-amplification of multiple polynucleotide assembly blocks, as the polynucleotide of interest is being assembled, not from individual rare or abundant blocks, but from common polynucleotide templates (ie: the polynucleotide contigs). Use of polynucleotide contigs as a common template allows a user to introduce quality control by amplification of specific or random regions of the polynucleotide contig, ensuring its integrity and full sequence coverage. Furthermore, use of polynucleotide contigs makes successful assembly of the polynucleotide of interest separate from the number of polynucleotide assembly blocks that need to be overlapped, and allows polynucleotide assembly block size and composition to focus on optimal design and assembly considerations.

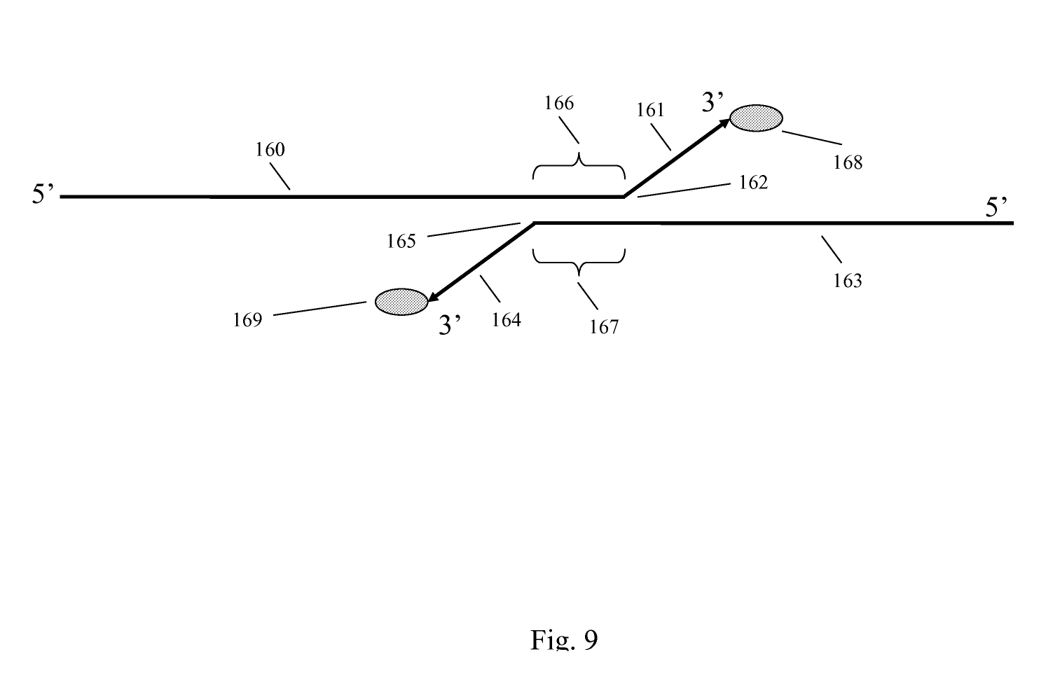

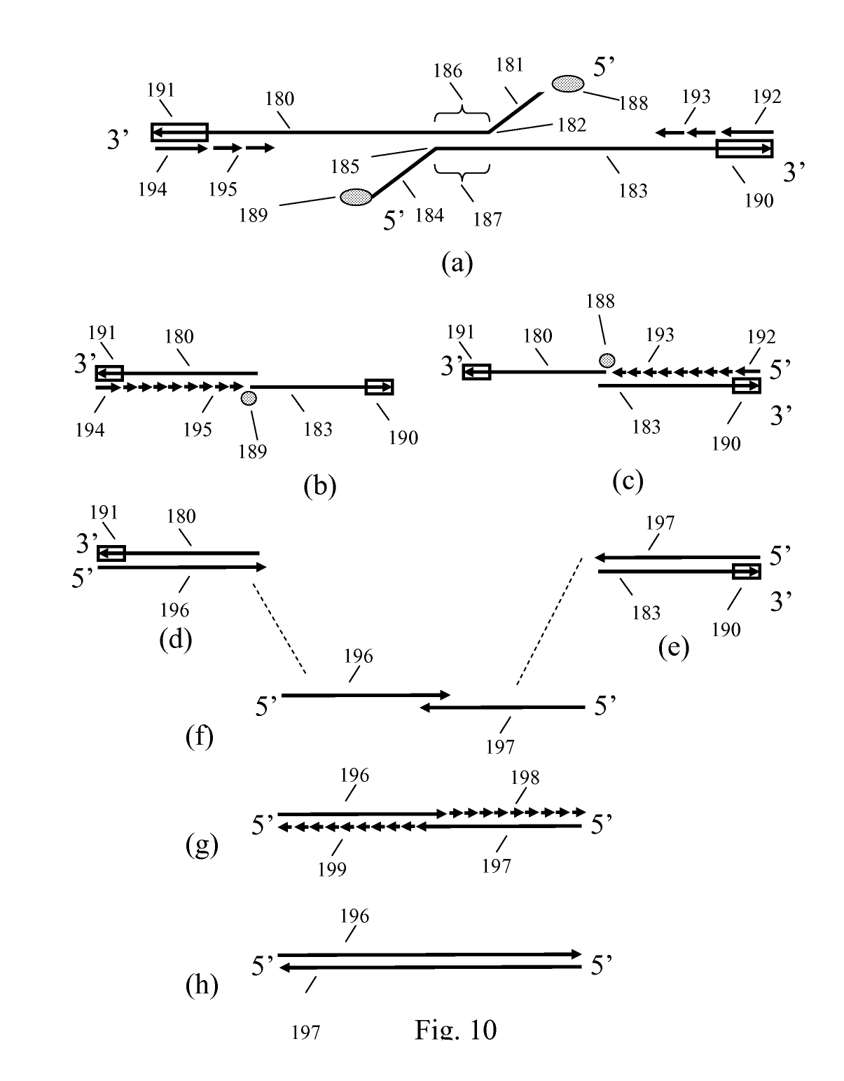

In other embodiments, the polynucleotide assembly block stock pool comprises polynucleotide assembly blocks from which the adaptors have not been removed. This avoids the need for adaptor sequences to contain restriction sites, avoids the need for restriction enzyme cleavage, and facilitates further amplification of the polynucleotide assembly block pool should such be desired. In these embodiments, the adaptor regions of the adjacent polynucleotide assembly blocks will extend beyond the overlap region upon hybridization of the overlap, as shown in FIGS. 9 and 10. The protruding adaptor regions may be removed during joining and amplification via the exonuclease activity of a polymerase having exonuclease activity, such as Taq polymerase. The exonuclease activity may be 3′ or 5′.

In one exemplary embodiment, removal of adaptors during block joining and amplification of a target polynucleotide is accomplished as illustrated in FIG. 9, by using a thermostable polymerase having 3′ to 5′ exonuclease activity for overlap PCR joining of adjacent polynucleotide assembly blocks. FIG. 9 illustrates the overlap PCR joining and accompanying exonuclease removal of adaptor sequences wherein block overlap region 166 of the sense strand of a first polynucleotide assembly block 160 having an adaptor sequence 161 extending in the 3′ direction from the block overlap 166, is hybridized with a reverse complementary block overlap 167 of the antisense strand of a second polynucleotide assembly block 163 having an adaptor sequence 164 extending in the 3′ direction from its block overlap 167. The thermostable polymerase 168, 169 having 3′ to 5′ exonuclease activity will first degrade the single stranded adaptor sequences 161, 164 extending in the 3′ direction beyond the hybridized region of overlap 166, 167. Upon degrading the single stranded adaptor sequences to the point of overlap 162, 165, the polymerase activity of the enzyme will result in chain extension from the sense strand of the first polynucleotide assembly block 160 in the 3′ direction, using the antisense strand of the second polynucleotide assembly block 163 as a template, and in extension from the antisense strand of the second polynucleotide assembly block 163 in the 3′ direction, using the sense strand of the second polynucleotide assembly block 160 as a template. In one non-limiting example the joining and 3′ adaptor sequence removal is performed in 15 μl comprising 2-2.5 ng of polynucleotide assembly module mixture, 1× iProof polymerase buffer, 200 uM dNTP, 0.6 μM each of the target sequence specific reverse primers complementary to the target specific primer sequences 170 and 171 (to facilitate amplification of the conjoined sequences), and 0.6u iProof polymerase, in 40 cycles each at 95° C. for 10 seconds, 55° C. for 20 seconds, and 72° C. for 15 seconds.

In another exemplary embodiment as illustrated in FIG. 10, the adaptor sequences are removed during overlap joining and amplification by using a thermostable polymerase having 5′ to 3′ exonuclease activity. In this exemplary embodiment, upon hybridization of the block overlap 186 of the antisense strand of the first polynucleotide assembly block 180 with the block overlap 187 of the sense strand of the second polynucleotide assembly block 183, single stranded 5′ terminal sequences complementary to the adaptor sequences will be exposed when present, and will be degraded by the 5′ to 3′ exonuclease activity of the polymerase as shown schematically in FIG. 10(a). The polymerase will extend each chain from the forward primers 192 and 194, and upon reaching the hybridized block overlap 182, 185 will degrade the opposite strand while continuing to extend the chain (FIGS. 10(b) and 10(c)). The resulting synthesized strands 196, 197 (FIGS. 10(d) and 10(e)) can then hybridize at the region of overlap (FIG. 10(f)), and chains are extended from the overlaps using the opposite sense strand as templates and the overlap as primers, producing the full length polynucleotide spanning the entire sequence between the forward and reverse primers (FIG. 10(g)). In one non-limiting embodiment, this joining and amplification is carried out according to PCR protocols familiar to a person having ordinary skill in the art, using a thermostable polymerase having 5′ to 3′ exonuclease activity such as Taq DNA polymerase. It will be apparent to a person having ordinary skill in the art that other choices of thermostable polymerase are possible for these and any other PCR amplification reactions, and the pertinent disclosures of the invention may be practiced using any thermostable polymerase having the activity or activities required for the operation of interest.

The present invention also provides kits, comprising one or more of the compositions of any of the various aspects and embodiments of the present invention. In various embodiments, the kits comprise one or more of a polynucleotide assembly block amplification pool, a polynucleotide assembly block stock pool, and or an oligonucleotide pool. The kits may further comprise any other reagents, buffers, etc. that are useful for carrying out the methods of the invention, such as those disclosed herein.

### EXAMPLES

### Example 1

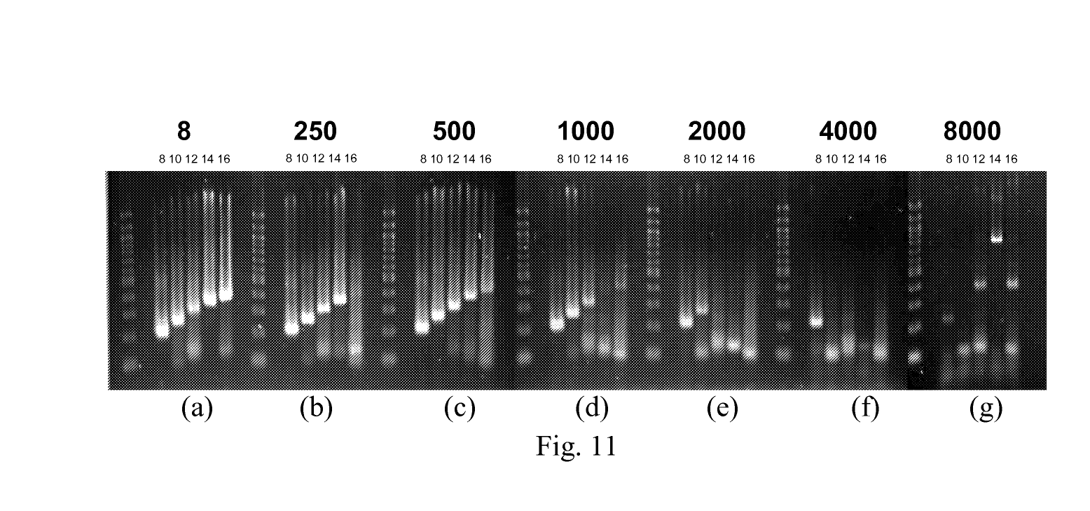

Example 1 shows that the methods, compositions, and kits disclosed herein enable assembly of polynucleotide assembly blocks from assembly oligonucleotides even when the assembly oligonucleotide pool contains a large number of diverse assembly oligonucleotide species. The influence of pool complexity on the specificity of gene assembly was determined using a set of individually-made assembly oligonucleotides, corresponding to several hundred genes of Bacillus anthracis. Equimolar amounts of 8, 250, 500, 100, 200, 400, and 8,000, respectively, of these assembly oligonucleotides were mixed to create assembly oligonucleotide pools of increasing complexities. To each pool was added a set of assembly oligonucleotides for assembling one of five anthrax genes, which provided a readout for confirming that assembly has occurred. Assembly oligonucleotides were provided for assembling a polynucleotide assembly blocks corresponding to each of the five readout genes from 8, 10, 12, 14, or 16 assembly oligonucleotides. The polynucleotide assembly blocks accordingly varied in length from about 200 bp to about 400 bp. Concentration of individual assembly oligonucleotides in all pools was maintained at 5 nM; 10 ul of an oligomix was used in a 50 ul assembly reaction containing 1× iProof polymerase buffer, 200 uM dNTP and 0.6u iProof polymerase. PCR was performed for 40 cycles each at 95° C. for 10 seconds, 55° C. for 20 seconds, and 72° C. for 15 seconds. Results of this experiment are shown in FIG. 11. FIGS. 11(a), 11(b), 11(c), 11(d), 11(e), 11(f), and 11(g) show gels corresponding to pool complexities of 8, 250, 500, 100, 200, 400, and 8,000, respectively. The lanes of each gel correspond to assembly of 8, 10, 12, 14, and 16 assembly oligonucleotides into a single polynucleotide assembly block as indicated. As FIGS. 11(a), (b), and (c) show, at pool complexities of up to 500, all polynucleotide assembly block sizes assemble, and blocks of 8, 10, 12, and 14 assembly oligonucleotides assemble at high quantity. As FIGS. 11(d), (e), (f), and (g) show, polynucleotide assembly blocks are successfully assembled from 8 assembly oligonucleotides even at very high pool complexities.

### Example 2

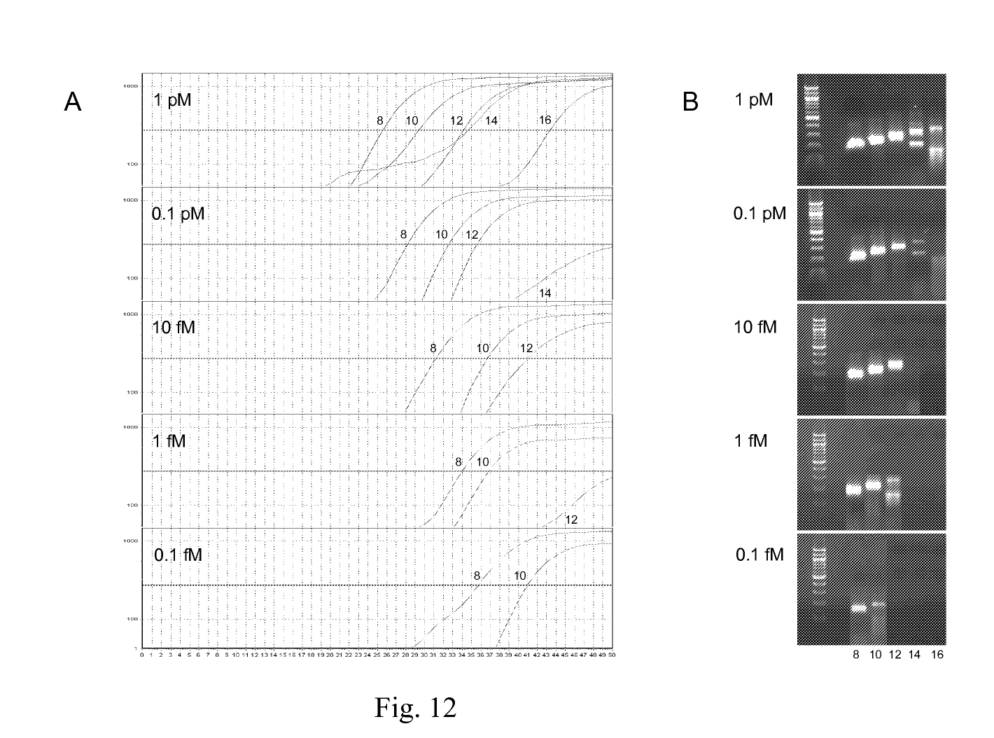

Example 2 shows that assembly oligonucleotides can be successfully assembled into polynucleotide assembly blocks even when the assembly oligonucleotides to be assembled are present at concentrations well below those currently practiced in the art. The five different anthrax gene blocks described in Example 1 were assembled in reactions at a series of assembly oligonucleotide concentrations. Reactions were set up with iProof DNA polymerase in 50 ul volumes and assembly was monitored by qPCR using a fluorescent readout (FIG. 12(A), where X-axis is cycle number, and Y-axis is relative fluorescence units of the PCR generated product). Products were also electrophoresed in an agarose gel to visualize (FIG. 12(B)). All five polynucleotide assembly blocks, assembled from 8, 10, 12, 14, and 16 assembly oligonucleotides, respectively, as shown in FIG. 12(B), were assembled at from assembly oligonucleotide pools containing 1 pM assembly oligonucleotides, although byproducts were also detected. Even in assembly reactions containing only 0.1 fM concentration of each assembly oligonucleotide, polynucleotide assembly blocks corresponding to lengths of 8 and 10 assembly oligonucleotides were successfully assembled. At 0.1 fM 50 ul of the assembling reaction contains 5 zeptomoles (10⁻²¹) or about 120 molecules of each oligo.

### Example 3

The methods disclosed herein for assembly of assembly oligonucleotides into polynucleotide assembly blocks were compared with assembly of the same oligonucleotides by a standard one step PCR procedure. Efficient and unbiased co-amplification of multiple polynucleotide assembly blocks in a single reaction was demonstrated. Blocks of approximately 250 bp, were assembled, each from ten fully overlapping 5′-phosphorylated assembly oligonucleotides using strict mismatch-exclusion temperature conditions. Lengths of assembly oligonucleotides varied from 36 to 56 bases, and the Tm of all overlap segments was 55±1° C. An annealing temperature of 58° C. was maintained throughout all LCR and PCR assemblies, which were performed using Taq ligase and iProof polymerase, respectively.

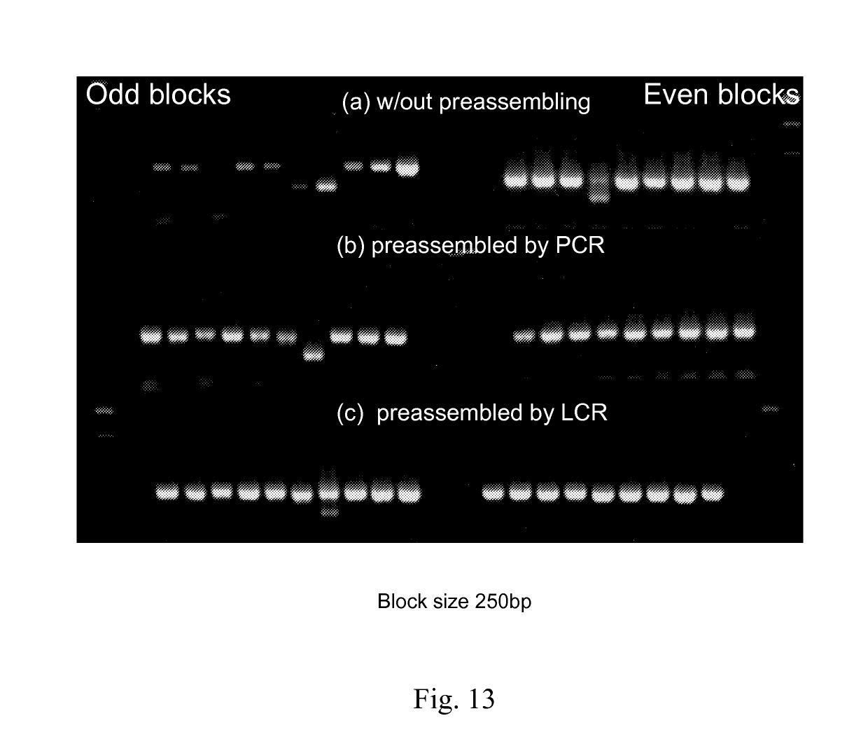

An assembly oligonucleotide pool comprising assembly oligonucleotides for the assembly of 20 distinct polynucleotide assembly blocks was assembled by each of three methods: (i) single-step PCR according to existing protocols; (ii) two-step PCR, comprising assembly of polynucleotide assembly blocks by spPCR followed by amplification of entire polynucleotide assembly block by PCR using the adaptor primers; and (iii) two-step LCR-PCR, comprising assembly of the polynucleotide assembly blocks by LCR, again followed by amplification of entire polynucleotide assembly block by PCR using the adaptor primers. The 20 polynucleotide assembly blocks were co-amplified in a single reaction and amounts of individual blocks in the pool were estimated by qPCR amplification of individual blocks. Results are presented in FIG. 13. Using the one step PCR method, the 20 co-amplified blocks were not generated with similar efficiency, and some were not detected at all (FIG. 13(a)). Using the two-step PCR method, 1 out of 20 blocks was not properly generated (FIG. 13(b)). The two step LCR-PCR method generated 20 high specificity products in well-balanced quantities (FIG. 13(c)).

### Example 4

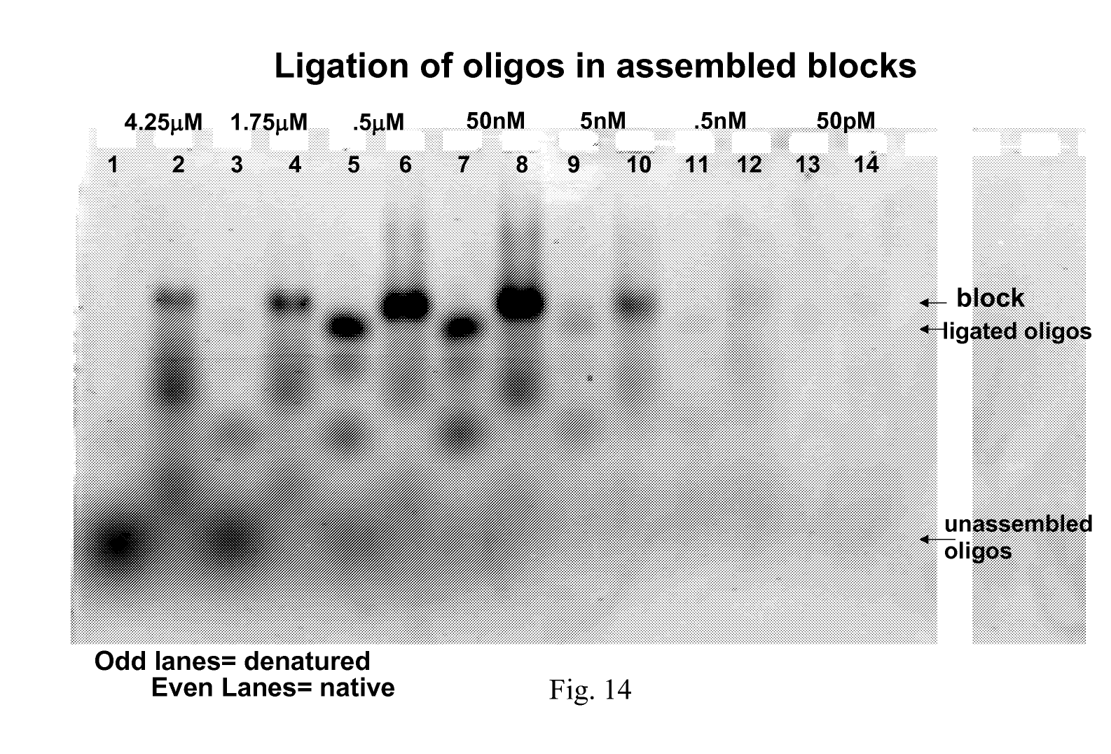

Efficiency of ligation and feasibility of the LCR preassembly step were demonstrated for assembly of polynucleotide assembly blocks each from eight assembly oligonucleotides by LCR using decreasing concentrations of assembly oligonucleotide ranging from 4.25 uM down to 50 pM as shown in FIG. 14. One of the assembly oligonucleotides was fluorescently labeled for subsequent sample detection. Native (even lines) and formamide denatured (odd lines) products of each LCR were loaded on an agarose gel and electrophoresis fractionated. Products were electrophoresed in an agarose gel with or without formamide denaturation immediately prior to loading. Similar amounts of DNA were loaded to facilitate visualization. Presence of a correct size high MW fragment in non-denatured samples (even lanes) indicates that blocks were appropriately assembled at all of the tested assembly oligonucleotide concentrations. Presence of a correct size high MW fragment in formamide denatured samples (odd lanes) indicates that after 40 cycles of LCR at all but the two highest tested concentrations, assembly oligonucleotides were covalently linked into blocks. Lack of detectable amount of covalently linked products at highest concentration is possibly due to the quantity of ligase used having been insufficient for ligating the large quantity of assembly oligonucleotides present.

### Example 5

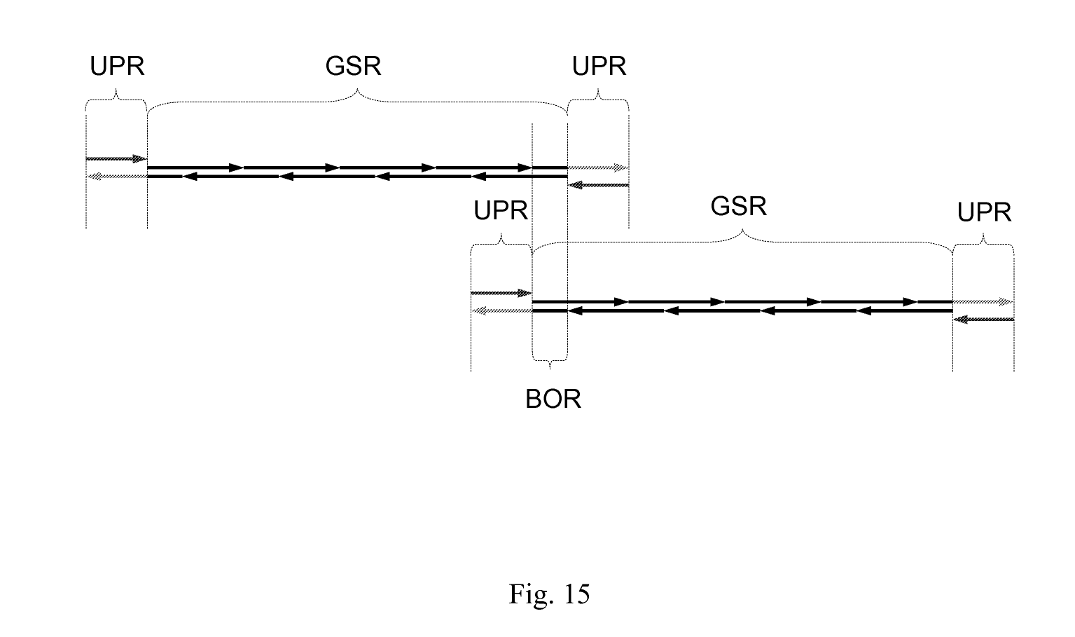

Twelve different primer/adaptor pairs were designed and evaluated based on (i) efficiency and uniformity of polynucleotide assembly block amplification, and (ii) the efficiency of the Mly I cleavage reaction used to remove the adaptors. For this evaluation, twelve variants of each of two overlapping polynucleotide assembly blocks, block A and block B, were designed and assembled, each variant incorporating one of a set of twelve different primers/adaptor sequences, but otherwise identical. To evaluate efficiency and uniformity of amplification each of the twelve variants of block A and each of the twelve variants of block B were amplified with a pair of corresponding primers. As diagrammed in FIG. 15, each of the blocks were assembled from eight internal assembly oligonucleotides spanning the gene specific region (GSR in FIG. 8) plus two terminal assembly oligonucleotides. Each terminal assembly oligonucleotide carried a sequence (UPR in FIG. 15) complimentary to the primer for block co-amplification. All overlap segments were designed to provide a normalized Tₘ of 55±1° C., fostering balanced amplification. A Mly I restriction site was included within the adaptor region, so that sequences outside the GSR could be removed by exposure to this type II restriction enzyme. Two adaptor sequence designs, one for block A and one for block B, were used to avoid unintended amplification of the short regions between adaptors of adjacent blocks, corresponding to the block overlapping region (BOR in FIG. 15). In one experiment, two distinct adaptor sequences were alternated for assignment to adjacent blocks, and two sets of block primers were used; blocks were designated as odd or even based on the adaptor sequence included in the primer. In a second experiment, only one set of adaptor sequences and block primers were used, and odd and even blocks were amplified in separate pools, which were then combined for assembly of the GSR from the two blocks.

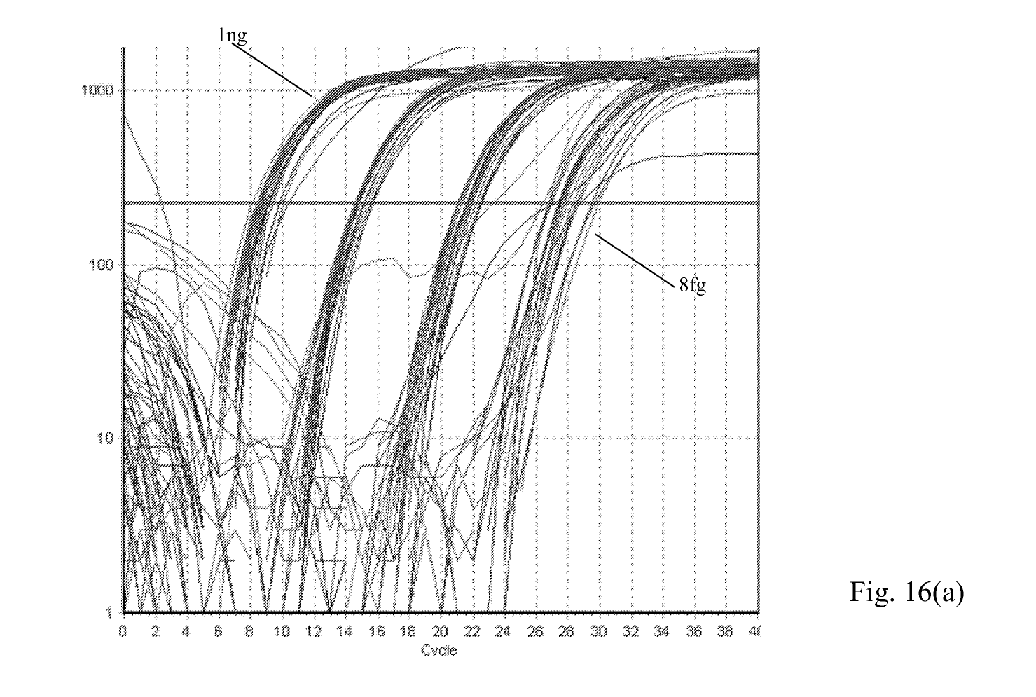

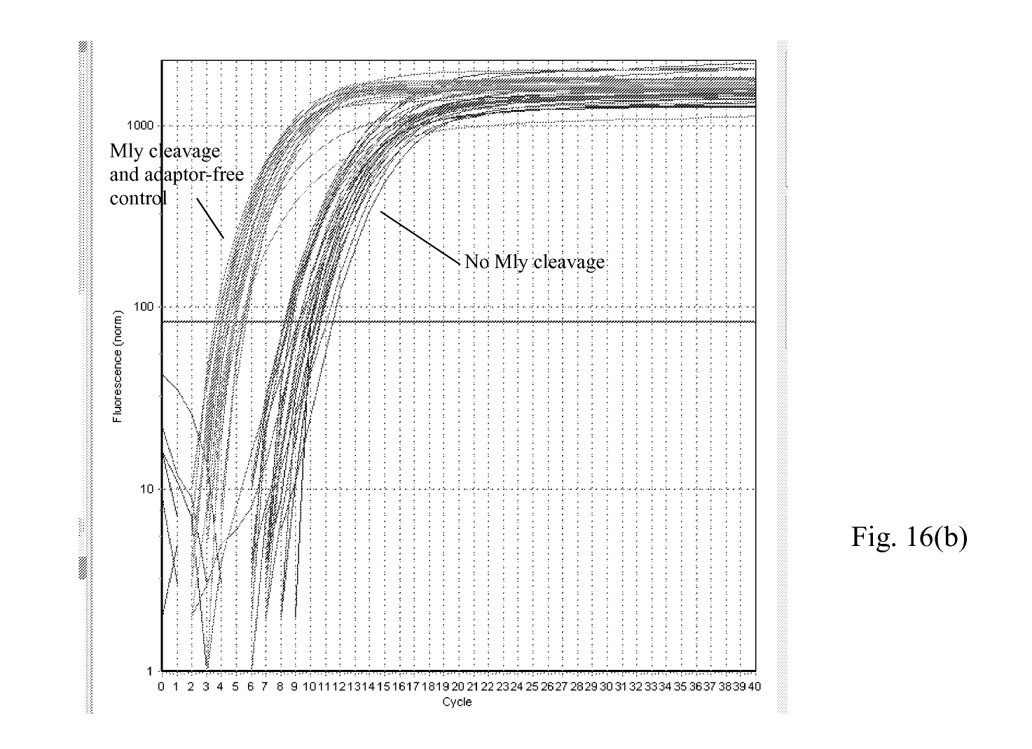

Efficiency of amplification was monitored by qPCR and results are presented in FIG. 16. Amplification efficiency was tested for four different starting amounts of template (1 ng, 20 pg, 400 fg, and 8 fg). All 12 primers performed approximately equally efficiently at the 1 ng-400 fg template quantities. The spread in number of cycles required for amplification was less than two cycles. At 8 fg, a greater spread was observed, indicating that some primers amplified at higher rates than others. No large differences between primers were detected, but primers A, D, E, F, G, H and L performed more robustly through the range of template concentrations than primers B, C, I, K and M. At the lowest concentration of template primers F, G, and H demonstrated the higher and more equally efficient amplification of both tested blocks.

Efficiency of the adaptor cleavage was evaluated by efficiency of block joining into a single product by overlapping PCR with and without MlyI digestion. The same sets of A and B blocks described above were used in this experiment. Blocks generated without adaptors were used as a 100% adaptor-free positive control. The merging reaction was monitored by qPCR and results are presented in FIG. 16b. Judging by efficiency of the overlapping reaction, all 12 tested adaptor sequences were removed from the blocks with approximately equal efficiency after incubation of 50 ng of the A and B block mixture for 2.5 hours at 37° C. in 1× Mly I restriction buffer supplemented with BSA and 10u of Mly I enzyme. Overlapping efficiency of the Mly I cleaved blocks was undistinguishable from that of the (control) blocks assembled without adaptors. As it is clear from FIG. 16b, blocks can be efficiently merged together even without adaptor cleavage, because of the exonuclease activity of the polymerase. An agarose gel analysis and sequencing evaluation of the products confirmed complete and proper removal of the adaptor sequences.

### Example 6

The 20 blocks of Example 3 were assembled from both column and chip synthesized oligos as indicated in Table I. Groups of three adjacent blocks were assembled into ˜600 bp fragments and pairs of 600 bp fragments were joined together into ˜1.2 kB fragments by overlapping PCR. All individual blocks and the overlapping products were cloned and random sets of clones sequenced. Products from standard gene assembly reaction using single step PCR amplification were similarly analyzed. The sequence analysis data are presented in Table 1. Frequency of flawless sequences among those assembled by the LCR/PCR method as disclosed herein was more than 10 times higher than the frequency of flawless sequences assembled from the same quality assembly oligonucleotides by single step PCR using existing methods [14] (Table I, first row) for target polynucleotides of similar size. Very similar type and frequencies of mutations were found in two sets of clones assembled by the methods disclosed herein from oligos of different quality, indicating that careful temperature design of the overlap segments and use of a discriminating annealing temperature both improves the success of block assembly, and renders the integrity of the sequence highly insensitive to the quality of the constituent oligos.

TABLE I
Accuracy of target polynucleotide sequence

| | Assembly | | | | Errors in | | |
|---|---|---|---|---|---|---|---|
| | oligo | Design and | | Flawless | imperfect | | |
| | synthesis | synthesis | Target | products (% of | products, per 1 kb | | |
| | method | protocol | polynucleotide size | ttl sequenced) | Ins | Dels | Subs |
| 1 | Single | Prior art | 671 bp | 3% | 1.1 | 3.6 | 2.5 |
| | column | | | | | | |
| 2 | Single | Invention | 215 bp | 58% | 0 | 2.5 | 0.5 |
| | column | | | | | | |
| 3 | Single | Invention | 310 bp | 60% | 0.4 | 1.0 | 0.1 |
| | column | | | | | | |
| 4 | Single | Invention | 469 bp | 16% | 0.1 | 4.7 | 0.9 |
| | column | | | | | | |
| 5 | Chip | Invention | 215 bp | 64% | 0 | 3.4 | 0.2 |
| 6 | Chip | Invention | 603 bp | 28% | 0 | 3.6 | 0.5 |
| 7 | Chip | Invention | 1192 bp | 10% | 0 | 2.6 | 0.8 |

All assembling reactions were performed in 1× iProof polymerase buffer supplemented with 200 uM dNTP, 1 nM of each of the gene assembling oligos, 0.6 μM each of the target sequence specific primers, and 0.6u iProof polymerase, in 40 cycles each at 95° C. for 10 seconds, 55° C. for 20 seconds, and 72° C. for 15-45 seconds, depending on the size of the product. Products in rows 2, 3, and 4 are single blocks of 3 different sizes, assembled from individual, column synthesized oligonucleotides. Products in rows 2 and 5 demonstrate comparative assembly efficiencies of target polynucleotides from individual versus chip synthesized oligonucleotides, with invention. Products in rows 6 and 7 are assembled blocks. Rows 2 and 6 compare products from high quality/quantity oligonucleotides and existing protocols versus low quality/quantity oligonucleotides and invention.

### Example 7

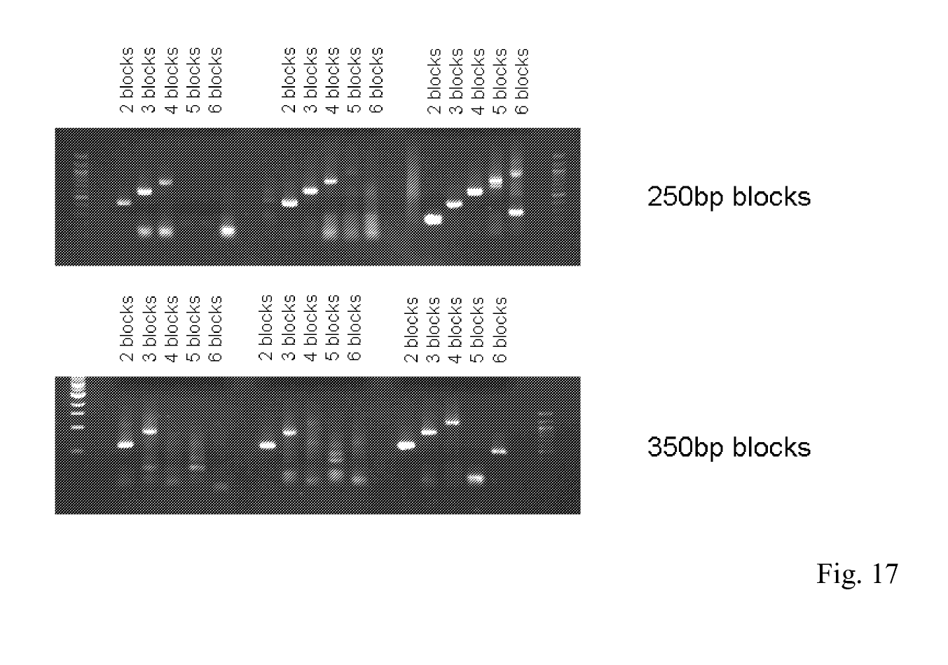

The efficiency of assembly three different target polynucleotide sequences from 2 to 6 polynucleotide assembly blocks was investigated using nominal block sizes of 250 and 350 base pairs assembled from 8 and 10 assembly oligonucleotides respectively. FIG. 17 shows that 5 blocks of 250 bp each, or 4 blocks of 350 bp each can be assembled in one reaction to produce 1.1 kb and 1.3 kb target polypeptides. Reaction was performed on 1 ng of each block in 1× iProof polymerase buffer supplemented 200 uM dNTP, 0.6 μM each of the target sequence specific primers, and 0.6u iProof polymerase, by 40 cycles each at 95° C. for 10 seconds, 55° C. for 20 seconds, and 72° C. for 45 seconds.

### Example 8

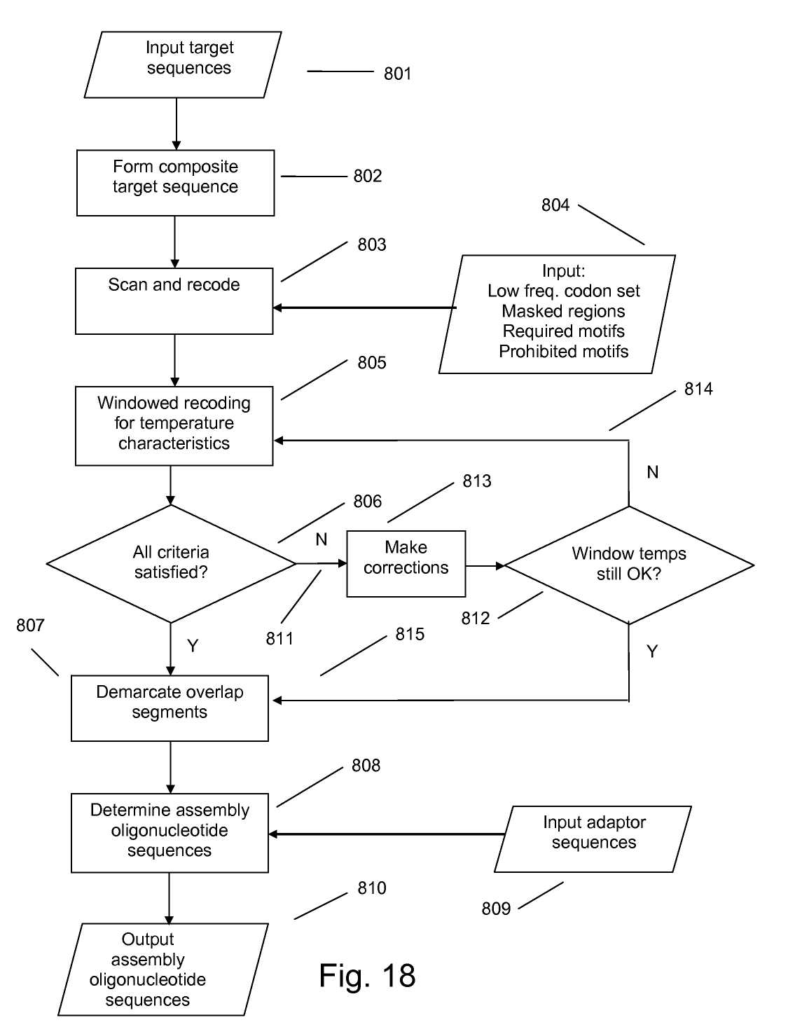

In a non-limiting exemplary embodiment, the sequences of assembly oligonucleotides for use in assembly of polynucleotide assembly blocks from which desired target polynucleotide sequences can be obtained are specified in accordance with the following steps, which are presented diagrammatically in FIG. 18:

1. The target polynucleotide sequences desired to be provided for are specified (block 801). The desired target sequence strings are abutted end-to-end, maintaining correct sense, to form a single composite target sequence for recoding, overlap segment design, and allocation to assembly oligonucleotides (block 802). A record is maintained of the boundaries of any non-coding regions in the composite sequence string. Any regions whose sequences are desired to be left unchanged are identified and their boundaries recorded (block 804). Any regions whose sequences are desired to conform to a particular motif or pattern are identified and the motif or pattern specified for each (block 804). Any sequence motifs desired to be avoided, such as restriction sequences, are specified (block 804).

2. The set of low-frequency codon types for the expression environment of interest is specified (block 804).

3. The combined sequence is scanned, any regions whose sequences are required to conform to a particular motif or pattern are made to so conform, and any low-frequency codon type occurring within any coding region is replaced by a randomly chosen acceptable codon type. Any regions matching sequence motifs desired to be avoided are identified; if any such region is a coding region, one or more codons are replaced by other acceptable codon types as necessary to disrupt the undesired motif, and if any such region is a non-coding region, one or more nucleotides are replaced with other nucleotides as necessary to disrupt the undesired motif (block 803).

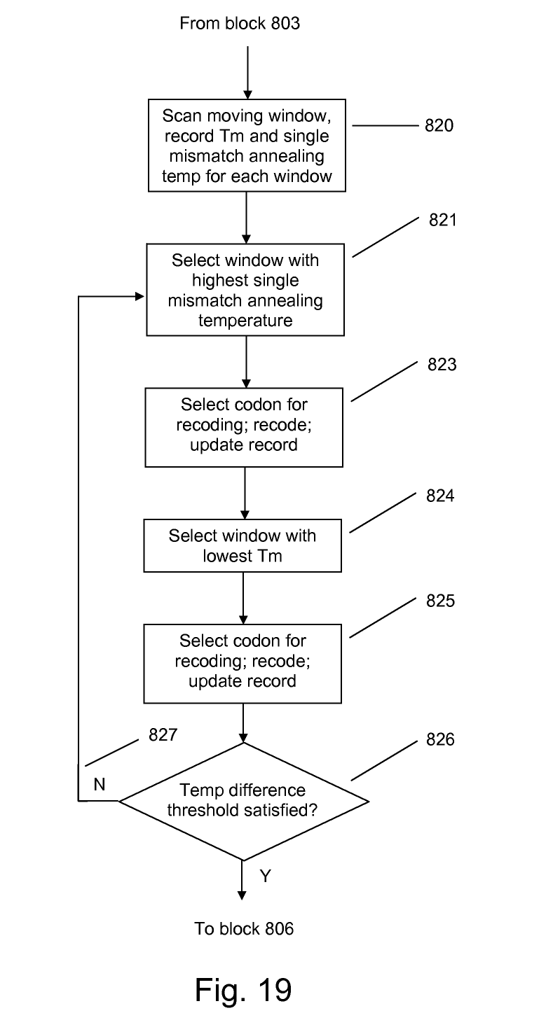

4. The composite sequence is again scanned within a moving window 25 nucleotides in length, which is advanced one nucleotide at a time, and the melting temperature and single-mismatch annealing temperature is estimated for the subsequence within the window at each advance (FIG. 18, block 805; FIG. 19, block 820). This is done for the entire combined sequence. The difference between the lowest melting temperature for any window and the highest single-mismatch annealing temperature for any window is iteratively improved by repeating the steps (diagrammed in FIG. 19) of
(a) selecting the window having the highest single-mismatch annealing temperature (block 821),
(b) selecting from within the selected window a codon (in the case of coding regions) or nucleotide (in the case of non-coding regions) for which there is an acceptable substitution whose effect would be to reduce the single-mismatch annealing temperature of the sequence within the window, making that substitution, and recomputing and updating the melting temperatures and single-mismatch annealing temperatures of all windows affected by the substitution (block 823);
(c) selecting the window having the lowest melting temperature (block 824); and
(d) selecting from within the selected window a codon (in the case of coding regions) or nucleotide (in the case of non-coding regions) for which there is an acceptable substitution whose effect would be to increase the melting temperature of the sequence within the window, making that substitution, and recomputing the melting temperatures and single-mismatch annealing temperatures of all windows affected by the substitution (block 825);
   until (block 826) the difference between the lowest melting temperature in any window and the highest single-mismatch annealing temperature in any window is greater than a specified threshold.

5. The entire recoded composite sequence is scanned (block 806) to ensure that no low-frequency codons remain, that any regions required to conform to a particular sequence or motif do so conform, that no regions required to be left unchanged have been disturbed, and that no undesired motifs have been introduced, making any necessary corrections (block 813). If any corrections are made, the temperature characteristics of any affected windows are recomputed (block 812). It is determined whether the difference between the lowest melting temperature in any window and the highest single-mismatch annealing temperature in any window remains below the desired threshold, and if not, the steps of the method are repeated from and including step 4 (arrow 814).

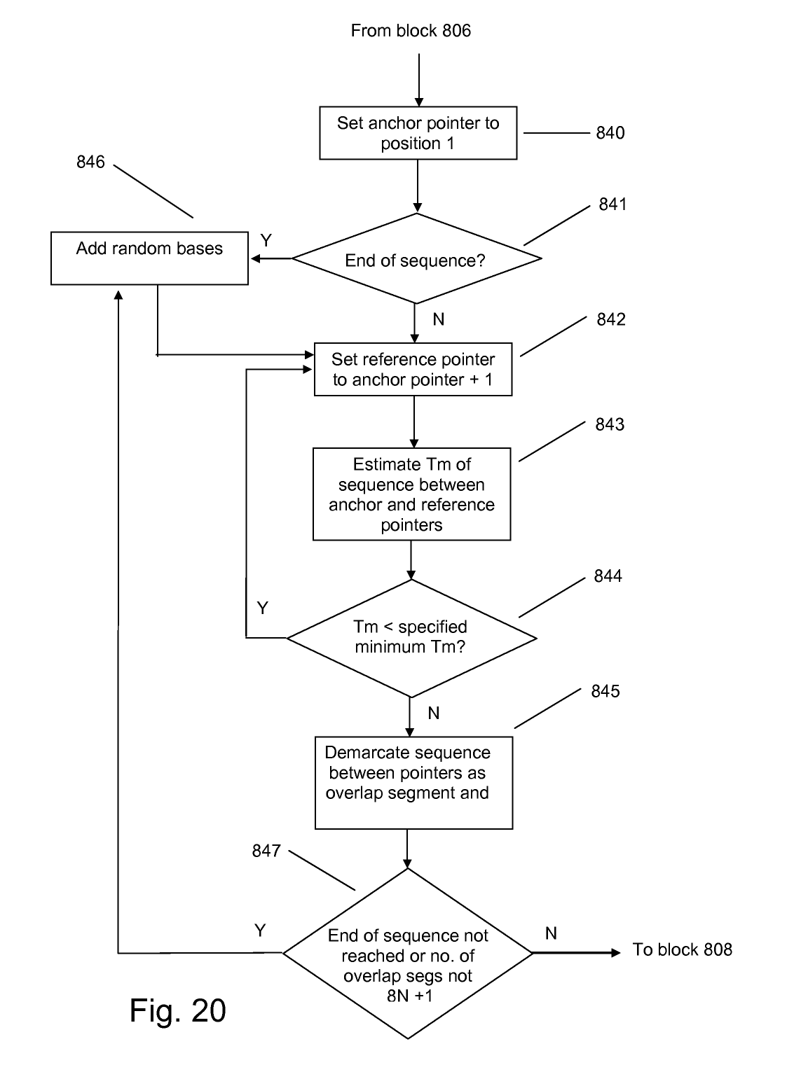

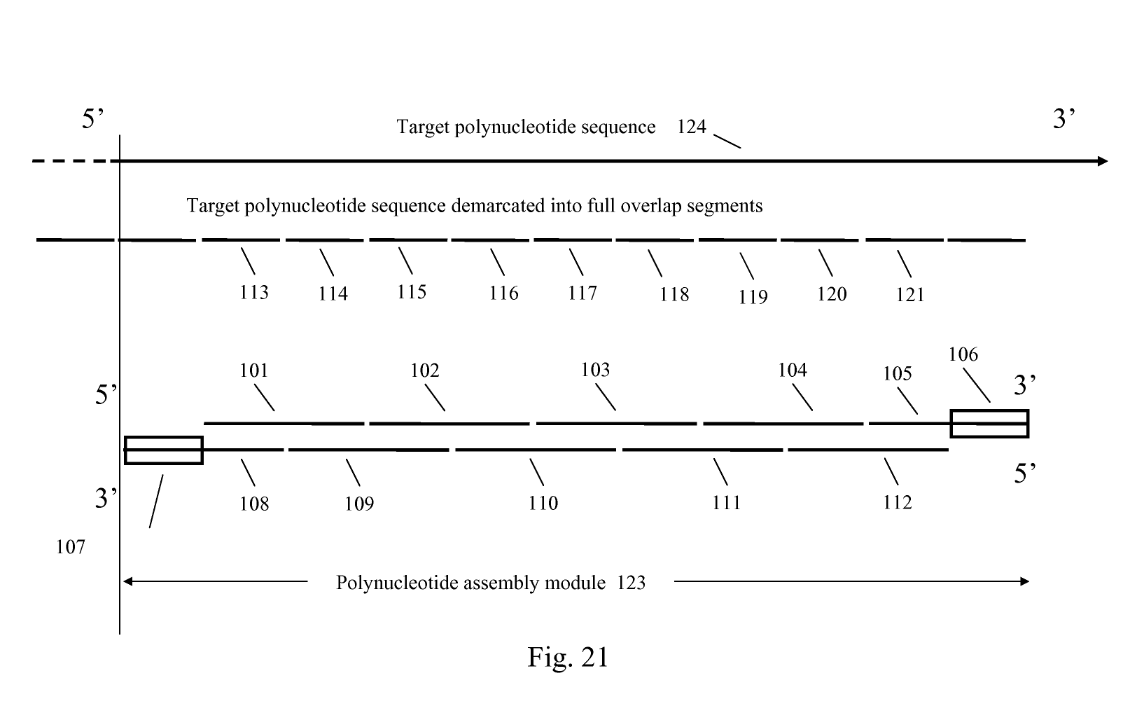

6. The recoded composite target sequence is analyzed for purposes of segregation into assembly oligonucleotides and polynucleotide assembly blocks (blocks 807 and 808). In this non-limiting example, this is accomplished as follows:
(a) Values are specified for the minimum melting temperature and maximum single-mismatch annealing temperature to be allowed for any overlap segment. The minimum melting temperature of any overlap segment in the assembly oligonucleotide block pool should be sufficiently high in comparison to the maximum single-mismatch annealing temperature of any overlap segment in the assembly oligonucleotide pool to provide the desired level of exclusion of incorrect sequences. In this exemplary embodiment, the minimum melting temperature is 57 degrees and the maximum single-mismatch annealing temperature is 59 degrees. (It will be apparent to persons having ordinary skill in the art that these temperatures are arbitrarily chosen for purpose of this example, and actual temperatures will depend upon many factors including without limitation the desired length of overlap segments, the sequence of overlap segments, and the annealing temperature to be used.)
(b) The recoded composite sequence is demarcated into a series of contiguous overlap segments that number one plus an integer multiple of eight as diagrammed in FIG. 20. (In this exemplary embodiment, a polynucleotide assembly block is assembled from ten assembly oligonucleotides, so one strand of a polynucleotide assembly block encompasses five conjoined assembly oligonucleotides, each internal assembly oligonucleotide encompasses two overlap segments, one of the resulting ten overlap segments is reserved for the 3′ terminal adaptor sequence, and each polynucleotide assembly block overlaps the adjacent polynucleotide assembly block by one overlap segment). FIG. 21 illustrates schematically the demarcation of a recoded combined sequence into polynucleotide assembly blocks, assembly oligonucleotides, and overlap segments as described for this exemplary embodiment. The demarcation of the recoded combined sequence into contiguous overlap segments is accomplished by scanning across the recoded composite sequence carrying out the following steps until the end of the recoded composite sequence is reached (FIG. 20):
(i) The “anchor pointer” is set to the first nucleotide position of the recoded composite target sequence (block 840).
(ii) The “reference pointer” is set to the locus immediately following that corresponding to the anchor pointer (block 842). (The subsequence beginning with the anchor pointer and ending with the reference pointer, inclusive, is referred to as the “reference subsequence” for purposes of this example.)
(iii) The estimated melting temperature and single-mismatch annealing temperature of the reference subsequence is computed (block 843).
(iv) If the estimated melting temperature of the reference subsequence is greater than or equal to the specified minimum melting temperature (block 844), the reference subsequence is demarcated as an overlap segment (block 845), the single-mismatch annealing temperature of the reference subsequence is estimated and recorded, the anchor pointer is set to the locus immediately following the reference pointer (block 842), and the preceding steps including this step are repeated from step 6(b)(ii) forward. If the estimated melting temperature of the reference subsequence is less than the specified minimum melting temperature (block 844), the reference pointer is advanced by one so that it corresponds to the next locus in the recoded composite sequence (block 842), and the preceding steps including this step are repeated from step 6(b)(iii) forward.
(c) If, as will ordinarily be the case, the number of overlap segments that have been demarcated when the reference pointer reaches the end of the recoded composite target sequence is not one plus an integer multiple of eight (block 847), then the recoded composite sequence is extended by adding nucleotides selected from the group consisting of A, T, C, and G to the 3′ end (block 846). Nucleotides are selected for adding to the composite sequence using a pseudorandom process constrained in such a way as to maintain uniform mismatch exclusion temperature characteristics in the added sequence. The demarcation of full overlap sequences is continued into the added subsequence until the number of overlap segments demarcated is one plus an integer multiple of eight. Similarly, if the reference pointer reaches the end of the recoded combined sequence before the estimated melting temperature of the last overlap segment required to raise the number of overlap segments to one more than an integer multiple of eight has exceeded the specified minimum melting temperature, the recoded composite target sequence is extended adding nucleotides to the 3′ end, again selecting the nucleotides to be added by a pseudorandom process constrained to maintain uniform mismatch exclusion temperature characteristics (block 846), until a last overlap segment exceeding the specified minimum melting temperature has been demarcated, and a total number of overlap segments equal to one more than an integer multiple of eight, each having an estimated melting temperature exceeding the specified minimum melting temperature, have been demarcated.

7. The sequences of the assembly oligonucleotides comprising the assembly oligonucleotide pool corresponding to the specified target polynucleotide sequences are determined (FIG. 18, block 808) by performing the following steps. (As used herein, the order of overlap segments is taken in the order of demarcation, in the 5′ to 3′ direction, so that the “first” overlap segment is the overlap segment comprising the 5′ end of the recoded composite target sequence, the “second” overlap segment is the next overlap segment demarcated proceeding in the 5′ to 3′ direction, and so on.)
(a) Adaptor sequences are specified for the sense and antisense strands of odd polynucleotide assembly blocks. Adaptor sequences are specified for the sense and antisense strands of even polynucleotide assembly blocks.
(b) Overlap segments corresponding to polynucleotide assembly blocks are determined as follows (as illustrated schematically in FIG. 18 with respect to the first polynucleotide assembly block of the composite target sequence):
   (i) The first polynucleotide assembly block comprises the first nine demarcated overlap segments.
   (ii) The remaining polynucleotide assembly blocks each comprise the last overlap segment corresponding to the previous polynucleotide assembly block, plus the next eight demarcated overlap segments in succession in the 3′ direction. Thus the second polynucleotide assembly block comprises the ninth through seventeenth demarcated overlap segments, and the last polynucleotide assembly block comprises the last nine demarcated overlap segments.
(c) For each block, the terminal assembly oligonucleotide of the sense strand comprises the sequence of the ninth (3′-most) demarcated overlap segment of the overlap segments allocated to the polynucleotide assembly block, conjoined to the sense strand adaptor sequence, which will be an odd adaptor sequence if the polynucleotide assembly block is an odd numbered block (numbering from 5′ to 3′ along the composite target sequence) and will be an even adaptor sequence if the polynucleotide assembly block is an even numbered block. The terminal assembly oligonucleotide of the antisense strand comprises the sequence of the antisense adaptor (again odd or even corresponding to whether the polynucleotide assembly block is an odd or even numbered block) conjoined to the reverse complement of the first overlap segment of the overlap segments allocated to the polynucleotide assembly block. The internal assembly oligonucleotides corresponding to the sense strand of the polynucleotide assembly block comprise the first and second, third and fourth, fifth and sixth, and seventh and eighth demarcated overlap segments, respectively, of the overlap segments allocated to the polynucleotide assembly block, each pair conjoined in the order as stated. The internal assembly oligonucleotides corresponding to the antisense strand of the polynucleotide assembly block comprise the reverse complements of the second and third, fourth and fifth, sixth and seventh, and eighth and ninth demarcated overlap segments, respectively, of the overlap segments allocated to the polynucleotide assembly block, each pair conjoined in the order as stated. The specification of sequences of assembly oligonucleotides from the demarcated overlap segments corresponding to a polynucleotide assembly block is illustrated schematically in FIG. 18, wherein the demarcated overlap segments 113, 114, 115, 116, 117, 118, 119, 120, and 121, their reverse complements, and the sense adaptor 106 and antisense adaptor 107 are conjoined into the terminal assembly oligonucleotide 105, specified by conjoining the ninth demarcated overlap segment 121 with the sense strand adaptor sequence 106; the terminal assembly oligonucleotide 108, specified by conjoining the antisense strand adaptor sequence 107 with the reverse complement of the first demarcated overlap segment 113; the sense strand internal assembly oligonucleotides 101, 102, 103, and 104, specified by conjoining the first eight demarcated overlap segments in pairs 113 and 114, 115 and 116, 117 and 118, and 119 and 120, respectively; and the antisense strand internal assembly oligonucleotides 109, 110, 111, and 112, specified by conjoining the reverse complements of the second through ninth demarcated overlap segments in pairs 114 and 115, 116 and 117, 118 and 119, and 120 and 121, respectively.

The present invention further provides computer readable storage media, for causing a processing device to automatically carry out any of the methods of the invention described above, including but note limited to the oligonucleotide design methods, the recoding methods, and the methods for assembling polynucleotides according to any embodiment disclosed herein. As used herein the term “computer readable medium” includes magnetic disks, optical disks, organic memory, and any other volatile (e.g., Random Access Memory (“RAM”)) or non-volatile (e.g., Read-Only Memory (“ROM”)) mass storage system readable by the CPU. The computer readable medium includes cooperating or interconnected computer readable medium, which exist exclusively on the processing system or be distributed among multiple interconnected processing systems that may be local or remote to the processing system.

It will be apparent to persons having ordinary skill in the art that many of the specifics of the method and algorithm described in the foregoing example, such as temperature thresholds and number of assembly oligonucleotides per polynucleotide assembly block, are arbitrarily chosen so as to provide a concrete example, and that many other values and/or choices would also be operable. The designation of various abstract entities such as the composite target polynucleotide sequence, demarcated overlap segments, sliding windows, anchor pointer, reference pointer, reference subsequences, and the like, as well as the division of the method into specific steps performed in a specific order, are for clarity of presentation, and it will be apparent to a person having ordinary skill in the art that literal designation of such entities is not required, nor it is necessary that the specific steps described be performed exactly as described; many other possible procedures, methods, or algorithms will produce similar results. The length of the moving window used for evaluating and making uniform the temperature characteristics of the composite target sequence may be any length; in a preferred embodiment, the length of the window is approximately equal to the desired approximate length of the overlap segments, which may be a length that results in the overlap segments having, on average, the desired mismatch-exclusion temperature characteristics. In the foregoing example, each successive overlap segment was demarcated at the first nucleotide causing the overlap segment melting temperature to exceed a specified threshold; it will be apparent to persons having ordinary skill in the art that various refinements are possible for conforming overlap segments more closely to the desired mismatch exclusion temperature characteristics, including without limitation performing further recoding on the overlap segments during or after demarcating them, and/or adjusting overlap segment boundaries iteratively. Various of the steps and/or substeps of the foregoing example may be omitted, combined, divided into additional substeps, and/or performed in another order, so as to tailor the method to the needs and goals of a particular application.

The specific description of the invention as disclosed herein should not be construed as limiting its scope, but rather as exemplifying certain embodiments thereof. Many other variations and applications of the methods, compositions, and kits disclosed herein are possible. The scope of the invention is determined not by the embodiments described herein but by the claims and their legal equivalents. Modifications of the methods, compositions, and kits disclosed herein that are obvious to those of skill in the fields of genetic engineering, molecular biology, or other relevant and/or related fields are intended to be within the scope of the invention.

### REFERENCES CITED

1. Au, L. C., et al., Gene synthesis by a LCR-based approach: High-level production of leptin-L54 using synthetic gene in Escherichia coli. Biochemical And Biophysical Research Communications, 1998. 248(1): p. 200-203.
2. Barany, F., Genetic Disease Detection and DNA Amplification Using Cloned Thermostable Ligase. PNAS, 1991. 88(1): p. 189-93.
3. Carr, P. A., et al., Protein-mediated error correction for de novo DNA synthesis. Nucleic Acids Research, 2004. 32(20).
4. Crea, R., et al., Chemical Synthesis Of Genes For Human Insulin. Proceedings Of The National Academy Of Sciences Of The United States Of America, 1978. 75(12): p. 5765-5769.
5. Ho, S, N., et al., Site-directed mutagenesis by overlap extension using the polymerase chain reaction. Gene, 1989. 77: p. 51-59.
6. Horton, R. M., et al., Engineering hybrid genes without the use of restriction enzymes: gene splicing by overlap extension. gene, 1989. 77: p. 61-68.
7. Itakura, K., et al., Expression In Escherichia-Coli Of A Chemically Synthesized Gene For Hormone Somatostatin. Science, 1977. 198(4321): p. 1056-1063.
8. Kim, C., et al., Progress in gene assembly from a MAS-driven DNA microarray. Microelectronic Engineering, 2006. 83(4-9): p. 1613-1616.
9. Kodumal, S. J., et al., Total synthesis of long DNA sequences: Synthesis of a contiguous 32-kb polyketide synthase gene cluster. PNAS, 2004. 101(44): p. 15573-15578.
10. Kong, D. S., et al., Parallel gene synthesis in a microfluidic device. Nucleic Acids Research, 2007. 35(8): p. e61.
11. Richardson, S. M., et al., GeneDesign: Rapid, automated design of multikilobase synthetic genes. Genome Research, 2006. 16(4): p. 550-556.
12. Richmond, K. E., et al., Amplification and assembly of chip-eluted DNA (AACED): a method for high-throughput gene synthesis. Nucleic Acids Research, 2004. 32(17): p. 5011-5018.
13. Sambrook, J. and D. W. Russell, Molecular Cloning: A Laboratory Manual. 3rd ed. Vol. II. 2001: Cold Spring Harbor Laboratory Press.
14. Stemmer, W. P. C., et al., Single-Step Assembly Of A Gene And Entire Plasmid From Large Numbers Of Oligodeoxyribonucleotides. Gene, 1995. 164(1): p. 49-53.
15. Sugimoto, N., et al., Thermodynamic Parameters To Predict Stability Of RNA/DNA Hybrid Duplexes. Biochemistry, 1995. 34(35): p. 11211-11216.
16. Tian, J. D., et al., Accurate multiplex gene synthesis from programmable DNA microchips. Nature, 2004. 432(7020): p. 1050-1054.
17. Vanden Heuvel, J. P., PCR Protocols in Molecular Toxicology. 1997: CRC.
18. Wiedmann, M., et al., Ligase Chain-Reaction (LCR)—Overview And Applications. PCR-Methods And Applications, 1994. 3(4): p. S51-S64.
19. Wu, G., et al., Simplified gene synthesis: A one-step approach to PCR-based gene construction. Journal Of Biotechnology, 2006. 124(3): p. 496-503.
20. Xia, T. B., et al., Thermodynamic parameters for an expanded nearest-neighbor model for formation of RNA duplexes with Watson-Crick base pairs. Biochemistry, 1998. 37(42): p. 14719-14735.
21. Xiong, A. S., et al., A simple, rapid, high-fidelity and cost-effective PCR-based two-step DNA synthesis method for long gene sequences. Nucleic Acids Research, 2004. 32(12).
22. Zhou, X. C., et al., Microfluidic PicoArray synthesis of oligodeoxynucleotides and simultaneous assembling of multiple DNA sequences. Nucleic Acids Research, 2004. 32(18): p. 5409-5417.
23. Breslauer, K. J., et al., Predicting DNA Duplex Stability From The Base Sequence. Proceedings Of The National Academy Of Sciences Of The United States Of America, 1986. 83(11): p. 3746-3750.
24. Owczarzy, R., Melting temperatures of nucleic acids: Discrepancies in analysis. Biophysical Chemistry, 2005. 117(3): p. 207-215.
25. Owczarzy, R., et al., Effects of sodium ions on DNA duplex oligomers: Improved predictions of melting temperatures. Biochemistry, 2004. 43(12): p. 3537-3554.
26. Owczarzy, R., et al., Predicting sequence-dependent melting stability of short duplex DNA oligomers. Biopolymers, 1997. 44(3): p. 217-239.
27. Allawi, H. T. and J. SantaLucia, Thermodynamics and NMR of internal GT mismatches in DNA. Biochemistry, 1997. 36(34): p. 10581-10594.
28. Owczarzy, R., F. J. Gallo, and A. S. Benight, Global comparison of published nearest-neighbor sequence dependent thermodynamic parameters. Biophysical Journal, 1997. 72(2): p. TH429-TH429.

We claim:

1. A method for polynucleotide assembly comprising:
(a) incubating, under hybridizing conditions, an oligonucleotide pool comprising a plurality of assembly oligonucleotides having mutually reverse complementary overlap segments, wherein the incubating results in assembly of a plurality of fully or partially double stranded polynucleotide assembly blocks, wherein the incubation is performed under conditions favoring hybridization of overlap segments that are exactly reverse complementary one to the other over hybridization of overlap segments with oligonucleotides whose sequences are not exactly reverse complementary thereto;
(b) amplifying the polynucleotide assembly blocks; and
(c) amplifying a polynucleotide of interest from one or more polynucleotide assembly blocks, wherein adjacent polynucleotide assembly blocks are joined at regions of mutual overlap as necessary to produce the polynucleotide of interest.

2. The method of claim 1 wherein one or more of the assembly oligonucleotides are recoded.

3. The method of claim 1 wherein a plurality of the assembly oligonucleotides are fully overlapping.

4. The method of claim 1 wherein a melting temperatures of all overlap segments of all assembly oligonucleotides corresponding to the polynucleotide assembly blocks from which a target polynucleotide is to be assembled are within a range of 0-10° C.

5. The method of claim 1 wherein a melting temperature of the overlap segment having the lowest melting temperature of all overlap segments of all assembly oligonucleotides corresponding to the polynucleotide assembly blocks from which a target polynucleotide is to be assembled is at least 1° C. higher than the single-mismatch annealing temperature of the overlap segment having the highest single-mismatch annealing temperature of all overlap segments of the same assembly oligonucleotides.

6. The method of claim 1 wherein assembly comprises assembly by ligation chain reaction.

7. The method of claim 1 wherein assembly comprises assembly by polymerase chain reaction.

8. The method of claim 1 wherein the amplifying a polynucleotide of interest from one or more polynucleotide assembly block comprises joining adjacent polynucleotide assembly blocks at regions of mutual overlap to form two or more polynucleotide contigs, and amplifying the polynucleotide of interest from the two or more polynucleotide contigs, wherein adjacent polynucleotide contigs are joined at regions of mutual overlap as necessary to produce the polynucleotide of interest.

9. A polynucleotide assembly block stock pool, comprising a plurality of polynucleotide assembly blocks having regions of common sequence.

10. The polynucleotide assembly block stock pool of claim 9, wherein the plurality of polynucleotide assembly blocks comprise adaptor sequences.

11. An oligonucleotide pool comprising a plurality of overlapping assembly oligonucleotides, wherein the overlap segments of the plurality of assembly oligonucleotides have lengths and compositions fostering preferential hybridization of overlap segments that are exactly reverse complementary one to the other over hybridization of overlap segments with oligonucleotides whose sequences are not exactly reverse complementary thereto.

12. A method for recoding a nucleic acid, comprising:
(a) determining low frequency codon types, maximum frequency codon types, and mid-frequency codon types for a given expression environment; and
(b) replacing all low-frequency codon types occurring within any coding regions of the nucleic acid with mid-frequency or maximum-frequency codon types.

13. The method of claim 12, further comprising making one or more acceptable substitutions that result in improvement of at least one characteristic of the nucleic acid.

14. The method of claim 12, wherein the expression environment comprises a mammalian cell, and wherein the low frequency codon types that are replaced are GCG, CGA, CGT, CTA, TTA, CCG, TCG, and ACG.

15. A method for specifying assembly oligonucleotide sequence, comprising:
(a) recoding a target polynucleotide sequence;
(b) allocating the target polynucleotide sequence to polynucleotide assembly blocks;
(c) determining boundaries of overlap segments in the target polynucleotide sequence or part thereof and its reverse complement; and
(d) determining boundaries of assembly oligonucleotide.
